(19) United States
(12) Patent Application Publication
(10) Pub. No.: US 2026/0008845 A1
(43) Pub. Date: Jan. 8, 2026

(54) BIOMARKERS FOR FIBROSIS TREATMENT USING ANTI-CLAUDIN-1 ANTIBODIES

(71) Applicant: Alentis Therapeutics AG, Allschwil (CH)

(72) Inventors: Elisa POLINI, Allschwil (CH); Roberto IACONE, Allschwil (CH); Geoffrey TEIXEIRA, Allschwil (CH); Alberto TOSO, Allschwil (CH); Markus KJ MEYER, Allschwil (CH)

(21) Appl. No.: 19/126,673

(22) PCT Filed: Nov. 14, 2023
(86) PCT No.: PCT/IB2023/000698

§ 371 Date: May 2, 2025

Related U.S. Application Data
(60) Provisional application No. 63/383,658, filed on Nov. 14, 2022.

(51) Int. Cl. C07K 16/28; A61K 39/00; G01N 33/68

(52) U.S. Cl. CPC C07K 16/28; G01N 33/6893; A61K 2039/505; A61K 2039/545; G01N 2333/70585; G01N 2333/70596; G01N 2800/7052

(57) ABSTRACT

The present disclosure relates to methods for identifying a subject or treating a subject with fibrosis or fibrosis progression. In some aspects, the method relates to measuring the levels of CD44, CD147 and/or osteopontin (SPP1) relative to a control level in order to guide a therapeutic regimen in a subject with fibrosis or fibrosis progression. The present disclosure also provides anti-Claudin-1 antibodies and fragments thereof and methods of use in diagnosing, providing prognosis or treating fibrosis or fibrosis progression.

### RELATED APPLICATIONS

This application claims priority to, and the benefit of, U.S. Provisional Application No. 63/383,658, filed Nov. 14, 2022, the contents of which are incorporated herein by reference in their entirety.

### REFERENCE TO AN ELECTRONIC SEQUENCE LISTING

The contents of the electronic sequence listing (ALNT-010_01WO_SeqList_ST26.xml; Size: 35,860 bytes; and Date of Creation: Nov. 14, 2023) are herein incorporated by reference in its entirety.

### FIELD

The disclosure relates to methods for identifying a subject or treating a subject with fibrosis or fibrosis progression. The method relates to measuring the levels of CD44, CD147 and/or ostcopontin (SPP1) relative to a control level in order to guide a therapeutic regimen in a subject with fibrosis or fibrosis progression. Further, the disclosure provides anti-Claudin-1 antibodies and antigen binding fragments thereof and methods of use in diagnosing, providing prognosis or treating fibrosis or fibrosis progression.

### BACKGROUND

Anti-Claudin-1 antibodies have been used to treat an array of diseases, such as hepatocellular carcinoma (e.g., U.S. Pat. No. 10,815,298), non-alcoholic fatty liver disease (e.g., U.S. Pat. No. 10,927,170), and fibrotic diseases such as renal fibrosis, pulmonary fibrosis, and skin fibrosis (e.g., WO 2021/094469 A1).

Human fibrotic diseases have a poor prognosis comparable with end-stage cancer. They represent an increasing cause of morbidity and mortality worldwide. Since fibrosis is a predominant feature of the pathology of a wide range of diseases across multiple organ systems, fibrotic disorders have been estimated to contribute to about 45% of all-cause mortality in the United States (Wynn, Nature Rev. Immunol., 2004, 4:583-594). The major health problem associated with fibrotic diseases is also due to our incomplete understanding of the underlying pathogenesis, the marked heterogeneity in the etiologies and clinical manifestations of fibrotic disorders, the absence of appropriate and fully validated biomarkers, and most importantly, the current void of effective disease-modifying therapeutic agents. At present, there are only two recently approved drugs specifically indicated for the treatment of fibrotic disease.

Therefore, a need exists to identify biomarkers that will improve treatment with anti-Claudin-1 antibodies (e.g., treatment of fibrotic diseases). Provided herein are methods and compositions addressing this need

### SUMMARY

The disclosure provides a method for identifying a subject having fibrosis that is suitable for therapy with an anti-Claudin-1 antibody or antigen binding fragment thereof, comprising the steps of: a) obtaining a biological sample a human subject; b) detecting the expression levels of one or more biomarkers selected from the group consisting of CD44, CD147, osteopontin (SPP1) and a combination thereof; c) comparing the detected expression level of the one or more biomarkers with a control level of the one or more biomarker; and d) identifying the human subject as a responder when the detected expression level of the one or more biomarkers is greater than the control level of CD44, CD147, or SPP1 expression, thereby identifying the human subject having fibrosis that is suitable for therapy with the anti-Claudin-1 antibody or antigen binding fragment thereof. In some aspects, the method further comprises: e) administering the anti-Claudin-1 antibody or antigen binding fragment thereof in an amount sufficient to alleviate a symptom of fibrosis when the subject is identified as a responder.

The disclosure provides a method of identifying fibrosis progression in a human subject that is suitable for therapy with an anti-Claudin-1 antibody or an antigen binding fragment thereof comprising the steps of: a) obtaining a biological sample a human subject; b) detecting the expression levels of one or more biomarkers selected from the group consisting of CD44,CD147, SPP1 and a combination thereof; c) comparing the detected expression level of the one or more biomarkers with a control level of the one or more biomarker; and d) identifying the human subject as a responder when the detected expression level of the one or more biomarkers is greater than the control level of CD44, CD147, or SPP1 expression, thereby identifying fibrosis progression in the human subject that is suitable for therapy with an anti-Claudin-1 antibody or an antigen binding fragment thereof. In some aspects. the method further comprises: e) administering the anti-Claudin-1 antibody or antigen binding fragment thereof in an amount sufficient to alleviate a symptom of fibrosis progression when the subject is identified as a responder.

The disclosure provides a method for treating a subject having fibrosis, the method comprising the steps of: a) obtaining a biological sample a human subject; b) detecting the expression levels of one or more biomarkers selected from the group consisting of CD44, CD147. SPP 1 and a combination thereof; c) comparing the detected expression level of the one or more biomarkers with a control level of the one or more biomarker; and d) identifying the human subject as a responder when the detected expression level of the one or more biomarkers is greater than the control level of CD44, CD147, or SPP1 expression, and e) administering the anti-Claudin-1 antibody or antigen binding fragment thereof in an amount sufficient to alleviate a symptom of fibrosis when the subject is identified as a responder, thereby treating the subject having fibrosis.

In some aspects, the biological sample is derived from blood, serum or tissue.

In some aspects, the control level of CD44 is about 200 ng/mL to about 400 ng/mL.

In some aspects, the control level of CD 147 is about 400 ng/mL to about 800 ng/mL.

In some aspects, the control level of SPP1 is about 20 ng/mL to about 80 ng/mL.

In some aspects, the subject has previously been administered with anti-Claudin-1 antibody or antigen binding fragment thereof.

In some aspects, the previous administration of anti-Claudin-1 antibody or antigen binding fragment thereof in the subject is administered at a first dose, and when the subject is identified as a responder, the subject is administered with at least a second dose of anti-Claudin-1 antibody or antigen binding fragment thereof.

In some aspects, the first dose and the second dose are equal. In some aspects, the second dose is greater than the first dose.

In some aspects. the first dose is about 0.3 mg/kg, 3 mg/kg. 10 mg/kg. 15 mg/kg or 20mg/kg. In some aspects, the first dose administered one time, two times, three times, four times or five times.

In some aspects, the second dose is about 0.3 mg/kg, 3 mg/kg, 10 mg/kg. 15 mg/kg or 20mg/kg. In some aspects, the second dose is administered one time, two times, three times, four times or five times.

In some aspects, the anti-Claudin-1 antibody or antigen binding fragment thereof comprises: a CDR H1 comprising the amino acid sequence of SEQ ID NO: 5, a CDR H2 comprising the amino acid sequence of SEQ ID NO: 6, a CDR H3 comprising the amino acid sequence of SEQ ID NO: 7, a CDR L1 comprising the amino acid sequence of SEQ ID NO: 8, a CDR L2 comprising the amino acid sequence of GA, and a CDR L3 comprising the amino acid sequence of SEQ ID NO: 10.

In some aspects, the anti-Claudin-1 antibody or antigen binding fragment thereof comprises a heavy chain variable region comprising the amino acid sequence of SEQ ID NO: 3 and a light chain variable region comprising the amino acid sequence of SEQ ID NO: 4.

In some aspects, the anti-Claudin-1 antibody or antigen binding fragment thereof comprises a heavy chain comprising the amino acid sequence of SEQ ID NO: 1 and a light chain comprising the amino acid sequence of SEQ ID NO. 2.

In some aspects. the anti-Claudin-1 antibody or antigen binding fragment thereof is a humanized antibody.

In some aspects, the anti-Claudin-1 antibody or antigen binding fragment thereof is administered intratumorally, intravenously, intraperitoneally, intramuscularly, intrathecally or subcutaneously.

In some aspects, the fibrosis is kidney fibrosis, lung fibrosis, or liver fibrosis.

In some aspects, the method comprises administering a therapeutically effective amount of at least one additional therapeutic agent, wherein the additional therapeutic agent is nintedanib or pirfenidone.

In some aspects, the anti-Claudin-1 antibody or antigen binding fragment thereof and the at least one additional therapeutic agent is administered concurrently or sequentially.

### BRIEF DESCRIPTION OF THE DRAWINGS

FIG. 1 is an exemplary schematic diagram showing the effects of anti-Claudin-1 monoclonal antibody treatment (Anti-CLDN1 mAb) on the complex of CLDN1, CD44 and CD147. Treatment with an anti-Claudin-1 results in downstream activation of metalloproteinases that cleave CD44 and CD 147 into its soluble form.

FIG. 2A is an exemplary fluorescent microscopy image showing co-localization of anti-Claudin-1 monoclonal antibody and CD44 in kidney fibrosis.

FIG. 2B is an exemplary immunohistochemistry image showing kidney fibrosis.

FIG. 3 is a bar graph showing soluble CD44 (sCD44) levels measured by ELISA on Day 15. in humans treated with various doses of anti-Claudin-1 monoclonal antibody (pre-dose, 0.3 mg/kg, 3 mg/kg, and 10 mg/kg).

FIG. 4 is a bar graph showing soluble CD147 (sCD147) levels measured by ELISA on Day 15, in humans treated with various doses of anti-Claudin-1 monoclonal antibody (pre-dose, 0.3 mg/kg, 3 mg/kg, and 10 mg/kg).

FIG. 5A is a bar graph showing plasma levels of human CD44 measured by ELISA in human patients treated with various doses of anti-Claudin-1 monoclonal antibody (placebo, 3 mg/kg, 10 mg/kg or 15 mg/kg). Plasma levels were tested at various time points Pre-Dose Day 1 (D1 PD), Day 22 (D22). Day 29 (D29), Day 32 (D32). and Day 57 (D57).

FIG. 5B is a graph showing the D57 plasma levels of human CD44 measured by ELISA in human patients treated with anti-Claudin-1 monoclonal antibody (placebo, 3 mg/kg, 10 mg/kg or 15 mg/kg). The y-axis represents the percent difference at D57 in comparison to pre-dose Day 1 (D1 PD) plasma levels for the same patients. **represents p-value<0.01 (ANOVA).

FIG. 6A is a bar graph showing plasma levels of human SPP1 measured by ELISA in human patients treated with various doses of anti-Claudin-1 monoclonal antibody (placebo, 3 mg/kg, 10 mg/kg or 15 mg/kg). Plasma levels were tested at various time points Pre-Dose Day 1 (D1 PD), Day 22 (D22), Day 29 (D29), Day 32 (D32), and Day 57 (D57).

FIG. 6B is a graph showing the D57 plasma levels of human SPP1 measured by ELISA in human patients treated with anti-Claudin-1 monoclonal antibody (placebo, 3 mg/kg, 10 mg/kg or 15 mg/kg). The y-axis represents the percent difference at D57 in comparison to pre-dose Day 1 (D1 PD) plasma levels for the same patients. *represents p-value<0.05 (ANOVA).

### DETAILED DESCRIPTION

Provided herein are methods of identifying and treating a human subject having fibrosis, that is suitable for therapy with an anti-Claudin-1 antibody or antigen binding fragment thereof. This is based in part on the observation that CD44. CD147, osteopontin (SPP1) are biomarkers associated with Claudin-1 target engagement in fibrosis. Without being bound by theory, the measurement of CD44, CD147 and/or SPP1 against a control level of CD44, CD147 and/or SPP1 can be used to determine the subset of subjects which would be responsive to anti-Claudin-1 antibody therapies. Accordingly, it would be advantageous to detect the expression levels of CD44, CD147 and/or SPP1 and compare these levels to a control level of CD44, CD147 and/or SPP1 in order to identify a subject having fibrosis, identify a subject with fibrosis progression, and/or guide a therapeutic dose or therapeutic regimen to alleviate a symptom of fibrosis and/or fibrosis progression using an anti-Claudin-1 antibody or antigen binding fragment thereof.

The disclosure provides a method for identifying a subject having fibrosis that is suitable for therapy with an anti-Claudin-1 antibody or antigen binding fragment thereof, comprising the steps of: a) obtaining a biological sample a human subject; b) detecting the expression levels of one or more biomarkers selected from the group consisting of CD44, CD147, osteopontin (SPP1) and a combination thereof; c) comparing the detected expression level of the one or more biomarkers with a control level of the one or more biomarker; and d) identifying the human subject as a responder when the detected expression level of the one or more biomarkers is greater than the control level of CD44, CD147, or SPP1 expression, thereby identifying the human subject having fibrosis that is suitable for therapy with the anti-Claudin-1 antibody or antigen binding fragment thereof. In some aspects, the method further comprises: c) administering the anti-Claudin-1 antibody or antigen binding fragment thereof in an amount sufficient to alleviate a symptom of fibrosis when the subject is identified as a responder.

The disclosure provides a method of identifying fibrosis progression in a human subject that is suitable for therapy with an anti-Claudin-1 antibody or an antigen binding fragment thereof comprising the steps of: a) obtaining a biological sample a human subject; b) detecting the expression levels of one or more biomarkers selected from the group consisting of CD44, CD147, SPP1 and a combination thereof; c) comparing the detected expression level of the one or more biomarkers with a control level of the one or more biomarker; and d) identifying the human subject as a responder when the detected expression level of the one or more biomarkers is greater than the control level of CD44, CD147, or SPP1 expression, thereby identifying fibrosis progression in the human subject that is suitable for therapy with an anti-Claudin-1 antibody or an antigen binding fragment thereof. In some aspects, the method further comprises: e) administering the anti-Claudin-1 antibody or antigen binding fragment thereof in an amount sufficient to alleviate a symptom of fibrosis progression when the subject is identified as a responder.

The disclosure provides a method for treating a subject having fibrosis, the method comprising the steps of: a) obtaining a biological sample a human subject; b) detecting the expression levels of one or more biomarkers selected from the group consisting of CD44, CD147, SPP1 and a combination thereof; c) comparing the detected expression level of the one or more biomarkers with a control level of the one or more biomarker; and d) identifying the human subject as a responder when the detected expression level of the one or more biomarkers is greater than the control level of CD44, CD147, or SPP1 expression, and e) administering the anti-Claudin-1 antibody or antigen binding fragment thereof in an amount sufficient to alleviate a symptom of fibrosis when the subject is identified as a responder, thereby treating the subject having fibrosis.

In some aspects. provided herein is a method of treating fibrosis comprising (a) administering an anti-Claudin-1 antibody to a subject; (b) determining a level of soluble CD44 in a test sample from the subject; and (c) comparing the level of soluble CD44 in the test sample to a level of soluble CD44 in a control sample. In some aspects, if the level of soluble CD44 in the test sample is not increased relative to level of soluble CD44 in the control sample, then the dose of the anti-Claudin-1 antibody being administered to the subject is increased. In some aspects, if the level of soluble CD44 in the test sample is increased relative to the control sample, then the dose of the anti-Claudin-1 antibody being administered to the subject is not increased.

In some aspects, provided herein is a method of treating fibrosis in a subject that has been determined to have a level of soluble CD44 in a test sample from the subject that is not increased relative to a level of soluble CD44 in a control sample. In some aspects, the subject has previously been administered an anti-Claudin-1 treatment, the method comprising administering an increased dose of the anti-Claudin-1 treatment to the subject.

In some aspects. provided herein is a method of monitoring fibrosis progression in a subject comprising (a) quantifying a level of soluble CD44 in a test sample from the subject, wherein the subject has previously been administered an anti-Claudin-1 treatment; and (b) comparing the level of soluble CD44 in the test sample to a level of soluble CD44 in a control sample. In some aspects, a level of soluble CD44 in the test sample that is not increased relative to the level of soluble CD44 in the control sample indicates that the subject is susceptible to an increased dose of the anti-Claudin-1 treatment.

In some aspects, provided herein is a method of monitoring fibrosis progression in a subject comprising (a) determining a level of soluble CD44 in a test sample from the subject, wherein the subject has previously been administered an anti-Claudin-1 antibody; and (b) comparing the level of soluble CD44 in the test sample to a level of soluble CD44 in a control sample. In some aspects, a level of soluble CD44 in the test sample that is not increased relative to the level of soluble CD44 in the control sample indicates that the subject is susceptible to an increased dose of the anti-Claudin-1 antibody.

In some aspects, provided herein is a method for treating a subject suffering from fibrosis, the method comprising the steps of determining whether the subject has increased levels of soluble CD44 by obtaining a test sample from the subject and comparing the level of soluble CD44 in the test sample to a control sample. In some aspects, the subject has previously been administered a dose of an anti-Claudin-1 antibody. In some aspects, if the subject has increased levels of soluble CD44 in the test sample relative to the control sample, then the subject is not administered an increased dose of the anti-Claudin-1 antibody. In some aspects, if the subject does not have increased levels of soluble CD44 in the test sample relative to the control sample, then the subject is administered an increased dose of the an anti-Claudin-1 antibody.

In some aspects, provided herein is a method for designing a customized therapy for a subject suffering from fibrosis which comprises (a) quantifying the level of soluble CD44 in a test sample from the subject; (b) comparing the level of soluble CD44 in the test sample obtained in step (a) with the level of soluble CD44 in a control sample, (c) determining that the subject has a not increased level of soluble CD44 with respect to the level of soluble CD44 in the control sample; and (d) administering an increased dose of the anti-Claudin-1 antibody. In some aspects. wherein if the level of soluble CD44 in the test sample obtained in step (a) is not increased relative to the level of soluble CD44 in the control sample, then the subject is susceptible to receive an increased dose of the anti-Claudin-1 antibody. In some aspects, the subject has previously been administered a dose of an anti-Claudin-1 antibody

In some aspects, provided herein is a method of classifying a subject suffering from fibrosis into a cohort and treating said subject, comprising (a) quantifying the level of soluble CD44 in a test sample from the subject; (b) comparing the level of soluble CD44 in the test sample obtained in step (a) with the level of soluble CD44 in a control sample; (c) determining that the subject has a not increased level of soluble CD44 with respect to the level of soluble CD44 in the control sample; (d) classifying said subject into a cohort based on the level of soluble CD44 in the test sample relative to the control sample; and (e) administering an increased dose of the anti-Claudin-1 antibody. In some aspects, the subject has previously been administered a dose of an anti-Claudin-1 antibody. In some aspects. if the level of soluble CD44 in the test sample obtained in step (a) is not increased relative to the level of soluble CD44 in the control sample, then the subject is susceptible to receive an increased dose of the anti-Claudin-1 antibody.

In some aspects, provided herein is a method of treating fibrosis comprising: (a) administering an anti-Claudin-1 antibody to a subject; (b) determining a level of soluble CD147 in a test sample from the subject; and (c) comparing the level of soluble CD147 in the test sample to a level of soluble CD147 in a control sample. In some aspects, if the level of soluble CD147 in the test sample is not increased relative to level of soluble CD147 in the control sample, then the dose of the anti-Claudin-1 antibody being administered to the subject is increased. In some aspects, if the level of soluble CD147 in the test sample is increased relative to the control sample, then the dose of the anti-Claudin-1 antibody being administered to the subject is not increased.

In some aspects, provided herein is a method of treating fibrosis in a subject that has been determined to have a level of soluble CD147 in a test sample from the subject that is not increased relative to a level of soluble CD147 in a control sample. In some aspects, the subject has previously been administered an anti-Claudin-1 treatment, the method comprising administering an increased dose of the anti-Claudin-1 treatment to the subject.

In some aspects, provided herein is a method of monitoring fibrosis progression in a subject comprising (a) quantifying a level of soluble CD147 in a test sample from the subject, wherein the subject has previously been administered an anti-Claudin-1 treatment; and (b) comparing the level of soluble CD147 in the test sample to a level of soluble CD147 in a control sample. In some aspects, a level of soluble CD147 in the test sample that is not increased relative to the level of soluble CD147 in the control sample indicates that the subject is susceptible to an increased dose of the anti-Claudin-1 treatment.

In some aspects, provided herein is a method of monitoring fibrosis progression in a subject comprising (a) determining a level of soluble CD147 in a test sample from the subject; and (b) comparing the level of soluble CD 147 in the test sample to a level of soluble CD147 in a control sample. In some aspects, the subject has previously been administered an anti-Claudin-1 antibody. In some aspects, a level of soluble CD147 in the test sample that is not increased relative to the level of soluble CD147 in the control sample indicates that the subject is susceptible to an increased dose of the anti-Claudin-1 antibody.

In some aspects, provided herein is a method for treating a subject suffering from fibrosis, the method comprising the steps of determining whether the subject has increased levels of soluble CD147 by obtaining a test sample from the subject and comparing the level of soluble CD147 in the test sample to a control sample. In some aspects, the subject has previously been administered a dose of an anti-Claudin-1 antibody. In some aspects, if the subject has increased levels of soluble CD147 in the test sample relative to the control sample, then the subject is not administered an increased dose of the anti-Claudin-1 antibody. In some aspects, if the subject does not have increased levels of soluble CD147 in the test sample relative to the control sample. then the subject is administered an increased dose of the an anti-Claudin-1 antibody.

In some aspects, provided herein is a method for designing a customized therapy for a subject suffering from fibrosis which comprises (a) quantifying the level of soluble CD147 in a test sample from the subject; (b) comparing the level of soluble CD147 in the test sample obtained in step (a) with the level of soluble CD147 in a control sample, (c) determining that the subject has a not increased level of soluble CD147 with respect to the level of soluble CD147 in the control sample; and (d) administering an increased dose of the anti-Claudin-1 antibody. In some aspects, the subject has previously been administered a dose of an anti-Claudin-1 antibody. In some aspects. if the level of soluble CD147 in the test sample obtained in step (a) is not increased relative to the level of soluble CD147 in the control sample, then the subject is susceptible to receive an increased dose of the anti-Claudin-1 antibody.

In some aspects, provided herein is a method of classifying a subject suffering from fibrosis into a cohort and treating said subject, comprising (a) quantifying the level of soluble CD147 in a test sample from the subject; (b) comparing the level of soluble CD147 in the test sample obtained in step (a) with the level of soluble CD147 in a control sample; (c) determining that the subject has a not increased level of soluble CD147 with respect to the level of soluble CD147 in the control sample; (d) classifying said subject into a cohort based on the level of soluble CD147 in the test sample relative to the control sample; and (e) administering an increased dose of the anti-Claudin-1 antibody. In some aspects, the subject has previously been administered a dose of an anti-Claudin-1 antibody. In some aspects, if the level of soluble CD147 in the test sample obtained in step (a) is not increased relative to the level of soluble CD147 in the control sample, then the subject is susceptible to receive an increased dose of the anti-Claudin-1 antibody.

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately. roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/;” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain aspects, the polypeptides can occur as single chains or associated chains.

The terms “administer,” “administering,” “administration,” and the like, as used herein, refer to the physical introduction of a composition comprising a therapeutic agent (e.g., combination of an anti-Claudin-1 antibody and/or an immune checkpoint inhibitor) to a subject, using any of the various methods and delivery systems known to those skilled in the art. Routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means treating fibrosis (e.g. kidney fibrosis, liver fibrosis, or lung fibrosis).

The terms “subject,” “patient,” “individual,” and “host,” and variants thereof are used interchangeably herein and refer to any mammalian subject, including without limitation. humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications. As used herein, the phrase “subject in need thereof” includes subjects, such as mammalian subjects, that would benefit from administration of a therapeutic agent, e.g., an anti-Claudin-1 antibody.

It is understood that wherever aspects are described herein with the language “comprising,” otherwise analogous aspects described in terms of “consisting of” and/or “consisting essentially of” are also provided.

As used herein, the terms “derived from” or “derivative” refer to a component that is isolated from or made using a specified molecule, or information (e.g., a nucleic acid sequence) from the specified molecule. For example, a polynucleotide sequence that is derived from another polynucleotide sequence can include a polynucleotide sequence that is identical or substantially similar to the polynucleotide sequence it derives from. In the case of polynucleotides, the derived species can be obtained by, for example, naturally occurring mutagenesis, artificial directed mutagenesis, or artificial random mutagenesis. The mutagenesis used to derive polynucleotides can be intentionally directed or intentionally random, or a mixture of each. The mutagenesis of a polynucleotide to create a different polynucleotide derived from the first polynucleotide can be a random event (e.g., caused by polymerase infidelity) and the identification of the derived polynucleotide can be made by appropriate screening methods known in the art. In some aspects, a polynucleotide sequence that is derived from a first polynucleotide sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to the first polynucleotide sequence, respectively, wherein the derived polynucleotide sequence retains the biological activity of the original polynucleotide.

The term “human Claudin-1 (or CLDN1)” refers to a protein having the sequence shown in NCBI Accession Number NP_066924.1, or any naturally occurring variants commonly found in HCV permissive human populations.

The term “antibody”, as used herein, refers to any immunoglobulin that contains an antigen binding site that immunospecifically binds an antigen. As such, the term antibody encompasses not only whole antibody molecules, but also antibody fragments as well as variants (including derivatives) of antibodies and of antibody fragments as long as the derivatives and fragments maintain specific binding ability. The term encompasses monoclonal antibodies and polyclonal antibodies. The term also covers any protein having a binding domain, which is homologous or largely homologous to an immunoglobulin-binding domain. These proteins may be derived from natural sources, or partly or wholly synthetically produced. The term “specific binding”, when used in reference to an antibody, refers to an antibody binding to a predetermined antigen. Typically, the antibody binds with an affinity of at least 1×10⁷ M¹, and binds to the predetermined antigen with an affinity that is at least two-fold greater than the affinity for binding to a non-specific antigen (e.g., BSA, casein).

The term “monoclonal antibody” or antigen-binding fragment thereof refers to a homogeneous antibody or antigen-binding fragment population involved in the highly specific recognition and binding of a single antigenie determinant, or epitope. This is in contrast to polyclonal antibodies that typically include different antibodies directed against different antigenic determinants. The term “monoclonal antibody” or antigen-binding fragment thereof encompasses both intact and full-length monoclonal antibodies as well as antibody fragments (such as Fab, Fab′, F(ab′)₂, Fv), single chain (scFv) mutants, fusion proteins comprising an antibody portion, and any other modified immunoglobulin molecule comprising an antigen recognition site. Furthermore, a “monoclonal antibody” or antigen-binding fragment thereof refers to such antibodies and antigen-binding fragments thereof made in any number of manners including but not limited to by hybridoma, phage selection, recombinant expression, and transgenic animals.

As used herein, the term “humanized antibody” refers to a chimeric antibody comprising amino acid residues from non-human hypervariable regions and amino acid residues from human framework regions (FRs). In particular, a humanized antibody comprises all or substantially all of at least one, typically two, variable domains. in which all or substantially all of the complementarity determining regions (CDRs) are those of a human antibody. A humanized antibody optionally may comprise at least a portion of an antibody constant region derived from a human antibody. A “humanized form” of an antibody, e.g., a non-human antibody, refers to an antibody that has undergone humanization.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system (i.e., fibrosis). An effective amount can be administered in one or more administrations.

### II. Anti-Claudin-1 Antibodies

Antibodies directed against human Claudin-1 have been previously described to treat Hepatitis C virus infection, hepatocellular carcinoma, and certain fibrotic diseases, such as lung fibrosis (see WO 2010/034812, WO 2016/146809, and WO 2021/094469). Anti-Claudin-1 antibodies that can be used in the practice of the present invention include any antibody raised against Claudin-1. Examples are disclosed in WO 2010/034812 and WO 2017/162678.

Other examples of suitable anti-Claudin-1 antibodies include those disclosed in European Patent No. EP 1 167 389, in U.S. Pat. No. 6,627,439, in international patent application published under No. WO 2014/132307, in international patent applications published under No. WO 2015/014659 and No. WO 2015/014357, and in Yamashita et al., J. Pharmacol. Exp. Ther., 2015, 353(1): 112-118.

Anti-Claudin-1 antibodies or antigen binding fragments thereof suitable for use in the present invention may be polyclonal antibodies or monoclonal antibodies.

Anti-Claudin-1 antibodies or antigen binding fragments thereof suitable for use according to the present invention may also be “humanized”, (e.g., sequence differences between rodent antibodies and human sequences can be minimized by replacing residues which differ from those in the human sequences by site-directed mutagenesis of individual residues or by grafting of entire regions or by chemical synthesis). Humanized antibodies can also be produced using recombinant methods. In the humanized form of the antibody, some, most or all of the amino acids outside the CDR regions are replaced with amino acids from human immunoglobulin molecules, while some, most or all amino acids within one or more CDR regions are unchanged. Small additions, deletions, insertions, substitutions or modifications of amino acids are permissible as long as they do not significantly modify the biological activity of the resulting antibody. Suitable human “replacement” immunoglobulin molecules include IgG1, IgG2, IgG2a, IgG2b, IgG3, IgG4, IgA, IgM, IgD or IgE molecules, and fragments thereof.

In some aspects, a humanized anti-Claudin-1 antibody for use according to the present invention includes those described in WO 2017/162678, which is incorporated by reference in its entirety. Exemplary sequences for the anti-Claudin-1 antibody or antigen binding fragment used in the methods of the disclosure are described in Table 1.

TABLE 1
Exemplary anti-Claudin-1 antibody sequences

| Description | Sequence | SEQ ID NO: |
|---|---|---|
| H1L1 - | EVQLVESGGGLVKPGGSLRLSCAASGFSFSSYGMNWVRQAPGK | SEQ ID NO: 1 |
| Heavy | GLEWVSSISPSGSYFYYADSVKGRFTISRDNAKNSLYLQMNSL | |
| Chain #1 | RAEDTAVYYCARLPGFNPPFDHWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSIGTQTYICNVNHKPSNTKVDK | |
| | RVEPKSCDKTHTCPPCPAPEFEGGPSVFLFPPKPKDTLMISRT | |
| | PEVTCVVVDVSHEDPEVKFNWYVDGVEVHNAKTKPREEQYNST | |
| | YRVVSVLTVLHQDWINGKEYKCKVSNKALPASIEKTISKAKGQ | |
| | PREPQVYTLPPSREEMTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPG | |
| H1L1 - | DIQMTQSPATLSVSPGERATLSCKASONVGGNVDWYQWKPGQA | SEQ ID NO: 2 |
| Light Chain | PRLLIYGASNRYTGIPARFRGSGSGTEFTLTISSLQSEDFAVY | |
| | YCLQYKNNPWTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| H1L1 - VH | EVQLVESGGGLVKPGGSLRLSCAASGFSFSSYGMNWVRQAPGK | SEQ ID NO: 3 |
| | GLEWVSSISPSGSYFYYADSVKGRFTISRDNAKNSLYLQMNSL | |
| | RAEDTAVYYCARLPGFNPPFDHWGQGTLVTVSS | |
| H1L1 - VL | DIQMTQSPATLSVSPGERATLSCKASQNVGGNVDWYQWKPGQA | SEQ ID NO: 4 |
| | PRLLIYGASNRYTGIPARFRGSGSGTEFTLTISSLOSEDFAVY | |
| | YCLQYKNNPWTFGQGTKVEIK | |
| H1L1/H3L3 - | GFSFSSYG | SEQ ID NO: 5 |
| CDR H1 | | |
| H1L1/H3L3 - | ISPSGSYF | SEQ ID NO: 6 |
| CDR H2 | | |
| H1L1/H3L3 - | ARLPGEFPPFDH | SEQ ID NO: 7 |
| CDR H3 | | |
| H1L1/H3L3 - | QNVGGN | SEQ ID NO: 8 |
| CDR L1 | | |
| H1L1/H3L3 - | GA | - |
| CDR L2 | | |
| H1L1/H3L3 - | LQYKNNPWT | SEQ ID NO: 10 |
| CDR L3 | | |
| H3L3 - | QVQLVESGGGVVQPGRSLRLSCLGSGFSFSSYGMNWVRQAPGK | SEQ ID NO: 11 |
| Heavy | GLEWVASISPSGSYFYYADSVKGRFTISRDNSKNTLYLQMTSL | |
| Chain | RAEDTAIYYCARLPGFNPPFDHWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | RVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQ | |
| | PREPQVYTIPPSREEMTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPG | |
| H3L3- | DIQMTQSPSSLSASVGDRVTITCKASQNVGGNVDWYQWKPGKA | SEQ ID NO: 12 |
| Light Chain | PKLLIYGASNRYTGVPDRFRGSGSGTDFTLTISSLQPEDVATY | |
| | YCLQYKNNPWTFGGGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| H3L3 - VH | QVQLVESGGGVVQPGRSLRLSCLGSGFSFSSYGMNWVRQAPGK | SEQ ID NO: 13 |
| | GLEWVASISPSGSYFYYADSVKGRFTISRDNSKNTLYLQMTSL | |
| | RAEDTAIYYCARLPGFNPPFDHWGQGTLVTVSS | |
| H3L3 - VL | DIQMTQSPSSLSASVGDRVTITCKASQNVGGNVDWYQWKPGKA | SEQ ID NO: 14 |
| | PKLLIYGASNRYTGVPDRFRGSGSGTDFTLTISSLQPEDVATY | |
| | YCLQYKNNPWTFGGGTKVEIK | |
| H1L1 - | EVQLVESGGGLVKPGGSLRLSCAASGFSFSSYGMNWVRQAPGK | SEQ ID NO: 25 |
| Heavy | GLEWVSSISPSGSYFYYADSVKGRFTISRDNAKNSLYLQMNSL | |
| Chain #2 | RAEDTAVYYCARLPGFNPPFDHWGQGTLVTVSSASTKGPSVFP | |
| | LAPSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | RVEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWINGKEYKCKVSNKALPAPIEKTISKAKGQ | |
| | PREPQVYTLPPSREEMTKNQVSLTCLVKGFYPSDIAVEWESNG | |
| | QPENNYKTTPPVLDSDGSFFLYSKLTVDKSRWQQGNVFSCSVM | |
| | HEALHNHYTQKSLSLSPG | |

In some aspects, the “H1L1” anti-Claudin-1 antibody or antigen binding fragment thereof comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

In some aspects, the “H1Li” anti-Claudin-1 antibody or antigen binding fragment thereof comprises a complementarity determining region (CDR) L1 comprising the amino acid sequence set forth in SEQ ID NO: 8, a CDR L2 comprising the amino acid sequence “Gly Ala”, and a CDR L3 comprising the amino acid sequence set forth in SEQ ID NO: 10.

In some aspects, the “H1L1” anti-Claudin-1 antibody or antigen binding fragment thereof comprises a complementarity determining region (CDR) H1 comprising the amino acid sequence set forth in SEQ ID NO: 5, a CDR H2 comprising the amino acid sequence set forth in SEQ ID NO: 6, a CDR H3 comprising the amino acid sequence set forth in SEQ ID NO: 7, a complementarity determining region (CDR) L1 comprising the amino acid sequence set forth in SEQ ID NO: 8, a CDR L2 comprising the amino acid sequence “Gly Ala”, and a CDR L3 comprising the amino acid sequence set forth in SEQ ID NO: 10.

In some aspects, the complementarity determining regions (CDRs) disclosed herein are defined according to IMGT®. However, it is appreciated that other methods of defining the CDRs in the art can also be used.

In some aspects, the “H1L1” anti-Claudin-1 antibody or antigen fragment thereof comprises a heavy chain variable region comprising the amino acid sequence of SEQ ID NO: 3.

In some aspects, the anti-Claudin-1 antibody or antigen fragment thereof comprises a VH comprising the amino acid sequence set forth in SEQ ID NO: 3 or SEQ ID NO: 13.

In some aspects, the anti-Claudin-1 antibody or antigen fragment thereof comprises a VH comprising an amino acid sequence at least 85%, 86%, 87%, 88%, 89%, 90%, 91%, 92%, 93%, 94%, 95%, 96%, 97%, 98%, or 99% identical to SEQ ID NO: 3. In some aspects, the anti-Claudin-1 antibody or antigen fragment thereof comprises a VH comprising an amino acid sequence at least 85%, 86%, 87%, 88%, 89%, 90%, 91%, 92%, 93%, 94%, 95%, 96%, 97%, 98%, or 99% identical to SEQ ID NO: 13.

In some aspects, the “H1L1” anti-Claudin-1 antibody or antigen fragment thereof comprises a light chain variable region comprising the amino acid sequence of SEQ ID NO: 4.

In some aspects, the anti-Claudin-1 antibody or antigen fragment thereof comprises a VL comprising the amino acid sequence set forth in SEQ ID NO: 4 or SEQ ID NO: 14.

In some aspects, the anti-Claudin-1 antibody or antigen fragment thereof comprises a VL comprising an amino acid sequence at least 85%, 86%, 87%, 88%, 89%, 90%, 91%, 92%, 93%, 94%, 95%, 96%, 97%, 98%, or 99% identical to SEQ ID NO: 4. In some aspects, the anti-Claudin-1 antibody or antigen fragment thereof comprises a VL comprising an amino acid sequence at least 85%, 86%, 87%, 88%, 89%, 90%, 91%, 92%, 93%, 94%, 95%, 96%, 97%, 98%, or 99% identical to SEQ ID NO: 14.

In some aspects, the anti-Claudin-1 antibody or antigen fragment thereof comprises a VH comprising the amino acid sequence set forth in SEQ ID NO: 3; and a VL comprising the amino acid sequence set forth in SEQ ID NO: 4. In some aspects, the “H1L1” anti-Claudin-1 antibody or antigen fragment thereof comprises a heavy chain variable region comprising the amino acid sequence of SEQ ID NO: 3 and a light chain variable region comprising the amino acid sequence of SEQ ID NO: 4.

In some aspects, the anti-Claudin-1 antibody or antigen fragment thereof comprises a VH comprising the amino acid sequence set forth in SEQ ID NO: 13; and a VL comprising the amino acid sequence set forth in SEQ ID NO: 14.

In some aspects, the anti-Claudin-1 antibody or antigen fragment thereof comprises a heavy chain comprising the amino acid sequence set forth in SEQ ID NO: 1, SEQ ID NO: 11, or SEQ ID NO: 25. In some aspects the “H1L1” anti-Claudin-1 antibody or antigen fragment thereof comprises the heavy chain comprising the amino acid sequence of SEQ ID NO: 1.

In some aspects, the anti-Claudin-1 antibody or antigen fragment thereof comprises a Heavy Chain comprising an amino acid sequence at least 85%, 86%, 87%, 88%, 89%, 90%, 91%, 92%, 93%, 94%, 95%, 96%, 97%, 98%, or 99% identical to SEQ ID NO: 1. In some aspects, the anti-Claudin-1 antibody or antigen fragment thereof comprises a Heavy Chain comprising an amino acid sequence at least 85%, 86%, 87%, 88%, 89%, 90%, 91%, 92%, 93%, 94%, 95%, 96%, 97%, 98%, or 99% identical to SEQ ID NO: 11. In some aspects, the anti-Claudin-1 antibody or antigen fragment thereof comprises a Heavy Chain comprising an amino acid sequence at least 85%, 86%, 87%, 88%, 89%, 90%, 91%, 92%, 93%, 94%, 95%, 96%, 97%, 98%, or 99% identical to SEQ ID NO: 25.

In some aspects, the anti-Claudin-1 antibody or antigen fragment thereof comprises a light chain comprising the amino acid sequence set forth in SEQ ID NO: 2 or SEQ ID NO: 12. In some aspects, the “H1L1” anti-Claudin-1 antibody or antigen fragment thereof comprises the light chain comprising the amino acid sequence of SEQ ID NO: 2.

In some aspects, the anti-Claudin-1 antibody or antigen fragment thereof comprises a light chain comprising an amino acid sequence at least 85%, 86%, 87%, 88%, 89%, 90%, 91%, 92%, 93%, 94%, 95%, 96%, 97%, 98%, or 99% identical to SEQ ID NO: 2.

In some aspects, the anti-Claudin-1 antibody or antigen fragment thereof comprises a light chain comprising an amino acid sequence at least 85%, 86%, 87%, 88%, 89%, 90%, 91%, 92%, 93%, 94%, 95%, 96%, 97%, 98%, or 99% identical to SEQ ID NO: 12.

In some aspects, the anti-Claudin-1 antibody or antigen fragment thereof comprises a heavy chain comprising the amino acid sequence set forth in SEQ ID NO: 1; and a light chain comprising the amino acid sequence set forth in SEQ ID NO: 2. In some aspects, the “H1L1” anti-Claudin-1 antibody or antigen fragment thereof comprises a heavy chain comprising the amino acid sequence set forth in SEQ ID NO: 1; and a light chain comprising the amino acid sequence set forth in SEQ ID NO: 2.

In some aspects, the anti-Claudin-1 antibody or antigen fragment thereof comprises a heavy chain comprising the amino acid sequence set forth in SEQ ID NO: 25; and a light chain comprising the amino acid sequence set forth in SEQ ID NO: 2.

In some aspects, the anti-Claudin-1 antibody or antigen fragment thereof comprises a heavy chain comprising the amino acid sequence set forth in SEQ ID NO: 11; and a light chain comprising the amino acid sequence set forth in SEQ ID NO: 12.

In some aspects, the six complementarity determining regions (CDRs) of the anti-Claudin-1 antibody or antigen fragment thereof are the same as those in the anti-Claudin-1 monoclonal antibody or antigen fragment thereof secreted by a hybridoma cell line deposited at the DSMZ on Jul. 29, 2008 under an Accession Number DSM ACC2938.

In some aspects, the heavy chain variable region (“VH”) and the light chain variable region (“VL”) of the anti-Claudin-1 antibody or antigen fragment thereof are the same as those in the anti-Claudin-1 monoclonal antibody or antigen fragment thereof secreted by a hybridoma cell line deposited at the DSMZ on Jul. 29, 2008 under an Accession Number DSM ACC2938.

In some aspects, the heavy chain and light chain of the anti-Claudin-1 antibody or antigen fragment thereof are the same as those in the anti-Claudin-1 monoclonal antibody or antigen fragment thereof secreted by a hybridoma cell line deposited at the DSMZ on July 29, 2008 under an Accession Number DSM ACC2938.

The humanized anti-Claudin-1 antibody or antigen fragment thereof may be a full monoclonal antibody or antigen fragment thereof having an isotope selected from the group consisting of IgG1, IgG2, IgG3 and IgG4. Alternatively, the humanized anti-Claudin-1 antibody or antigen fragment thereof may be a fragment of a monoclonal antibody or antigen fragment thereof selected from the group consisting of Fv, Fab, F(ab′)₂, Fab′, dsFv, scFv, sc(Fv)₂ and diabodies.

Anti-Claudin-1 antibodies (or biologically active variants or fragments thereof) suitable for use according to the present invention may be functionally linked (e.g., by chemical coupling, genetic fusion, non-covalent association or otherwise) to one or more other molecular entities. Methods for the preparation of such modified antibodies (or conjugated antibodies) are known in the art (see, for example, “Affinity Techniques. Enzyme Purification: Part B”. Methods in Enzymol., 1974, Vol. 34, Jakoby and Wilneck (Eds.), Academic Press: New York, NY, and Wilchek and Bayer, Anal. Biochem., 1988, 171:1-32). Preferably, molecular entities are attached at positions on the antibody molecule that do not interfere with the binding properties of the resulting conjugate, e.g., positions that do not participate in the specific binding of the antibody to its target.

The antibody molecule and molecular entity may be covalently, directly linked to each other. Or, alternatively, the antibody molecule and molecular entity may be covalently linked to each other through a linker group. This can be accomplished by using any of a wide variety of stable bifunctional agents well known in the art, including homofunctional and heterofunctional linkers.

In some aspects, an anti-Claudin-1 antibody (or a biologically active fragment thereof) for use according to the present invention is conjugated to a detectable agent. Any of a wide variety of detectable agents can be used, including, without limitation, various ligands, radionuclides (e.g., 3H, 125I, 131I, and the like), fluorescent dyes (e.g., fluorescein isothiocyanate, rhodamine, phycoerytherin, phycocyanin, allophycocyanin, o-phthalaldehyde and fluorescamine), chemiluminescent agents (e.g., luciferin, luciferase and acquorin), microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

Other molecular entities that can be conjugated to an anti-Claudin-1 antibody of the present invention (or a biologically active fragment thereof) include, but are not limited to, linear or branched hydrophilic polymeric groups, fatty acid groups, or fatty ester groups.

Thus, in the practice of the present invention, anti-Claudin-1 antibodies can be used under the form of full length antibodies, biologically active variants or fragments thereof, chimeric antibodies, humanized antibodies, and antibody-derived molecules comprising at least one complementarity determining region (CDR) from either a heavy chain or light chain variable region of an anti-Claudin-1 antibody, including molecules such as Fab fragments, F(ab′)₂ fragments, Fd fragments, Fabc fragments, Sc antibodies (single chain antibodies), diabodies, individual antibody light single chains, individual antibody heavy chains, chimeric fusions between antibody chains and other molecules, and antibody conjugates, such as antibodies conjugated to a therapeutic agent or a detectable agent. Preferably, anti-Claudin-1 antibody-related molecules according to the present invention retain the antibody's ability to bind its antigen, in particular the extracellular domain of Claudin-1.

### Chimeric Antigen Receptors

Chimeric antigen receptor (CAR) T-cell therapy, or CAR T-cell therapy, is a cancer treatment, based on the use of T cells genetically engineered to express a synthetic receptor that binds a tumor antigen. Engineered CAR T cells are expanded in vitro and infused into the patient's body to attack and destroy chemotherapy-resistant cancer.

The term “chimeric antigen receptor” (CAR) refers to molecules that combine a binding domain against a component present on the target cell, for example an antibody-based specificity for a desired antigen (e.g., a tumor antigen, such as CLDN1) with a T cell receptor-activating intracellular domain to generate a chimeric protein that exhibits a specific anti-target cellular immune activity.

A “signal transducing domain” or “signaling domain” of a CAR, as used herein, is responsible for intracellular signaling following the binding of an extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector function signal and directs the cell to perform a specialized function. Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. In some cases, signaling domains comprise two distinct classes of cytoplasmic signaling sequences, those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Exemplary ITAMs include those derived from TCRzeta, FcRgamma, FcRbeta, FcRepsilon, CD3gamma, CD3delta, CD3epsilon, CD5, CD22, CD79a, CD79b and CD66d. In some aspects, the signal transducing domain of the CAR can comprise the CD3zeta signaling domain (SEQ ID NO: 19).

CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and heavy chain variable fragments of a monoclonal antibody joined by a flexible linker. This molecule is joined to an intracellular signaling molecule, comprising one or more intracellular signaling domains that mediate T-cell activation. The signaling domains for first generation CARs are derived from the cytoplasmic region of the CD3zeta or the Fe receptor gamma chains (or the intracellular signaling domain of another immune-receptor-tyrosine-based-activation-motif [ITAM]-containing protein). First generation CARs have been shown to successfully redirect T-cell cytotoxicity. However, they failed to provide prolonged expansion and anti-tumor activity in vivo. Signaling domains from co-stimulatory molecules, as well as transmembrane and binge domains have been added to form CARs of second, third, and fourth generations. Second generation chimeric receptors also incorporate a co-stimulatory endodomain (e.g., 4-1BB/CD35). Third generation CARs containing multiple co-stimulatory signaling modules. Fourth-generation CARs were generated by adding IL-12 to the base of the second-generation constructs, and are known as T cell redirected for universal cytokine-mediated killing (TRUCKs). TRUCKs augment T-cell activation and activate and attract innate immune cells to eliminate antigen-negative cancer cells in the targeted lesion. Therapeutic trials in humans using CAR T-cell therapy have shown some success. For example, CAR redirected T cells specific for the B cell differentiation antigen CD 19 have shown dramatic efficacy in the treatment of B cell malignancies, while TCR-redirected T cells have shown benefits in patients suffering from solid cancer. Stauss et al, describe strategies to modify therapeutic CARs and TCRs, for use in the treatment of cancer, for example, to enhance the antigen-specific effector function and limit toxicity of engineered T cells (Current Opinion in Pharmacology 2015, 24:113-118).

In some aspects of the present invention includes a chimeric antigen receptor (CAR) which is specific for Claudin-1, which is expressed on the surface of cancer cells. In some aspects of the present invention, a CAR as described herein comprises an extracellular target-specific binding domain, a transmembrane domain, an intracellular signaling domain (such as a signaling domain derived from CD3zeta or FcRgamma), and/or one or more co-stimulatory signaling domains derived from a co-stimulatory molecule, such as, but not limited to, 4-1BB. In some aspects, the CAR includes a hinge or spacer region between the extracellular binding domain and the transmembrane domain, such as a CD8alpha hinge. In some aspects, the chimeric antigen receptor (CAR) comprises an extracellular target-specific binding domain, which is an anti-Claudin single chain antibody (scFv), and may be a murine, human or humanized scFv. Single chain antibodies may be cloned from the V region genes of a hybridoma specific for a desired target. A technique which can be used for cloning the variable region heavy chain (VH) and variable region light chain (VL) has been described, for example, in Orlandi et al., PNAS, 1989; 86:3833-3837. Thus, in some aspects, a binding domain comprises an antibody-derived binding domain but can be a non-antibody derived binding domain. An antibody-derived binding domain can be a fragment of an antibody or a genetically engineered product of one or more fragments of the antibody, which fragment is involved in binding with the antigen.

In some aspects, the CARs of the present invention may comprise a linker between the various domains, added for appropriate spacing and conformation of the molecule. For example, in some aspects, there may be a linker between the binding domain VH or VL which may be between 1-10 amino acids long. In some aspects, the linker between any of the domains of the chimeric antigen receptor may be between 1-20 or 20 amino acids long. In this regard, the linker may be 1, 2, 3. 4, 5, 6, 7. 8, 9, 10, 11, 12, 13, 14, 15, 16, 17. 18, 19 or 20 amino acids long. In some aspects, the linker may be 21, 22, 23, 24, 25, 26, 27, 28, 29 or 30 amino acids long. Ranges including the numbers described herein are also included herein, e.g., a linker 10-30 amino acids long.

In some aspects, linkers suitable for use in the CAR described herein are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G) n, glycine-serine polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR can include linkers that are all or partially flexible, such that the linker can include a flexible linker as well as one or more portions that confer less flexible structure to provide for a desired CAR structure. Specific linkers include (G4S)n linkers, wherein n=1-3. In some aspects, the linker comprises the amino acid sequence of SEQ ID NO: 20 or SEQ ID NO: 21.

The binding domain of the CAR may be followed by a “spacer,” or, “hinge,” which refers to the region that moves the antigen binding domain away from the effector cell surface to enable proper cell/cell contact, antigen binding and activation (Patel et al., Gene Therapy, 1999; 6:412-419). The hinge region in a CAR is generally between the transmembrane (TM) and the binding domain. In some aspects, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type 1 membrane proteins such as CD8alpha, CD4, CD28 and CD7, which may be wild-type hinge regions from these molecules or may be altered. In some aspects, the hinge region comprises a CD8alpha hinge (SEQ ID NO: 22).

The “transmembrane” region or domain is the portion of the CAR that anchors the extracellular binding portion to the plasma membrane of the immune effector cell, and facilitates binding of the binding domain to the target antigen. The transmembrane domain may be a CD3 zeta transmembrane domain, however other transmembrane domains that may be employed include those obtained from CD8alpha, CD4, CD28, CD45, CD9, CD16, CD22, CD33, CD64, CD80, CD86, CD134, CD137, and CD154. In some aspects, the transmembrane domain is the transmembrane domain of CD137. In some aspects, the transmembrane domain comprises the amino acid sequence of SEQ ID NO: 23. In some aspects, the transmembrane domain is synthetic in which case it would comprise predominantly hydrophobic residues such as leucine and valine.

The “intracellular signaling domain” or “signaling domain” refers to the part of the chimeric antigen receptor protein that participates in transducing the message of effective CAR binding to a target antigen into the interior of the immune effector cell to elicit effector cell function, e.g., activation, cytokine production. proliferation and cytotoxic activity, including the release of cytotoxic factors to the CAR-bound target cell, or other cellular responses elicited with antigen binding to the extracellular CAR domain. The term “effector function” refers to a specialized function of the cell. Effector function of the T cell, for example, may be cytolytic activity or help or activity including the secretion of a cytokine. Thus, the terms “intracellular signaling domain” or “signaling domain,” used interchangeably herein, refer to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the, “signal transduction domain.” and is typically derived from portions of the human CD3 or FcRγ chains.

It is known that signals generated through the T cell receptor alone are insufficient for full activation of the T cell and that a secondary, or costimulatory signal is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling motifs which are known as immunoreceptor tyrosine-based activation motif or ITAMs.

Examples of ITAM containing primary cytoplasmic signaling sequences that are of particular use in the invention include those derived from TCRzeta, FcRgamma, FcRbeta, CD3gamma, CD3delta, CD3epsilon, CD5, CD22, CD79a, CD79b and CD66d. In one some aspects, the intracellular signaling domain of the anti-BCMA CARs described herein are derived from CD3zeta. In some aspects, the signaling domain comprises the amino acid sequence of SEQ ID NO: 19.

As used herein, the term, “costimulatory signaling domain,” or “costimulatory domain”, refers to the portion of the CAR comprising the intracellular domain of a costimulatory molecule. Costimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD3zeta and 4-IBB, other costimulatory domains are contemplated for use with the CARs described herein. The inclusion of one or more co-stimulatory signaling domains may enhance the efficacy and expansion of T cells expressing CAR receptors. The intracellular signaling and costimulatory signaling domains may be linked in any order in tandem to the carboxyl terminus of the transmembrane domain. In some aspects, the costimulatory domain comprises the amino acid sequence of SEQ ID NO: 24.

In some aspects, the anti-Claudin-1 CARs of the present disclosure comprise any of the elements of Table 2.

TABLE 2
CAR elements

| Description | Sequence | SEQ ID NO: |
|---|---|---|
| CD3zeta | RVKFSRSADAPAYKOGQNQLYNELNLGRREEYDVLDKRRGRD | SEQ ID NO: 19 |
| signaling | PEMGGKPRRKNPQEGLYNELQKDKMAEAYSEIGMKGERRRGK | |
| domain | GHDGLYQGLSTATKDTYDALHMQALPPR | |
| (G4S) linker 1 | GGGGSGGGGSGGGGS | SEQ ID NO: 20 |
| linker 2 | GSTSGSGKPGSGEGSTKG | SEQ ID NO: 21 |
| CD8alpha | TTTPAPRPPTPAPTIASQPLSLRPEACRPAAGGAVHTRGLDF | SEQ ID NO: 22 |
| hinge | ACD | |
| TM domain | IYIWAPLAGTCGVLLLSLVITLYC | SEQ ID NO: 23 |
| costimulatory | KRGRKKLLYIFKQPFMRPVQTTQEEDGCCRFPEEEEGGCEL | SEQ ID NO: 24 |
| domain | | |

Although scFv-based CARs engineered to contain a signaling domain from CD3 or FcRgamma have been shown to deliver a potent signal for T cell activation and effector function, they are not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from CD28, CD137, CD134 and CD278) may more effectively direct antitumor activity as well as increased cytokine secretion, lytic activity, survival and proliferation in CAR expressing T cells in vitro, and in animal models and cancer patients (Milone et al., Molecular Therapy. 2009; 17: 1453-1464; Zhong et al. Molecular Therapy, 2010:18: 413-420: Carpenito et al., PNAS, 2009; 106:3360-3365).

In some aspects, the anti-Claudin-1 CAR of the invention comprises (a) an anti-Claudin-1 binding domain (e.g., an scFv having binding regions (e.g., CDRs or variable domains) from any one or more of the sequences identified in Table 1) (b) a hinge region derived from human CD8alpha, (c) a human CD8alpha transmembrane domain, and (d) a human T cell receptor CD3 zeta chain (CD3) intracellular signaling domain, and optionally one or more costimulatory signaling domains, e.g., 4-1BB. In some aspects, the different protein domains are arranged from amino to carboxyl terminus in the following order: an anti-Claudin-1 binding domain, a hinge region and a transmembrane domain. The intracellular signaling domain and optional co-stimulatory signaling domains are linked to the transmembrane carboxy terminus in any order in tandem to form a single chain chimeric polypeptide. In some aspects, a nucleic acid construct encoding an anti-Claudin-1 CAR is a chimeric nucleic acid molecule comprising a nucleic acid molecule comprising different coding sequences, for example, (5′ to 3′) the coding sequences of an anti-Claudin-1 scFv, a human CD8alpha-hinge, a human CD8alpha transmembrane domain and a CD3zeta intracellular signaling domain. In some aspects, a nucleic acid construct encoding an anti-Claudin-1 CAR is a chimeric nucleic acid molecule comprising a nucleic acid molecule comprising different coding sequences, for example, (5′ to 3′) the coding sequences of an anti-Claudin-1 scFv, a human CD8alpha-hinge, a human CD8alpha transmembrane domain, a 4-1BB co-stimulatory domain, and a CD3zeta co-stimulatory domain.

In some aspects, the polynucleotide encoding the CAR described herein is inserted into a vector. For expression of an anti-Claudin-1 CAR, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences, These elements may be selected as appropriate by a person of ordinary skill in the art, as described above. For example, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

The CARs of the present invention are introduced into a host cell using transfection and/or transduction techniques known in the art. As used herein, the terms, “transfection,” and, “transduction.” refer to the processes by which an exogenous nucleic acid sequence is introduced into a host cell. The nucleic acid may be integrated into the host cell DNA or may be maintained extrachromosomally. The nucleic acid may be maintained transiently or may be a stable introduction. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection. protoplast fusion, retroviral infection, and biolistics. Transduction refers to the delivery of a gene(s) using a viral or retroviral vector by means of viral infection rather than by transfection.

As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably.

In some aspects, the CAR of the present invention is introduced and expressed in immune effector cells so as to redirect their specificity to a target antigen of interest, e.g. Claudin-1.

The present invention provides methods for making the immune effector cells which express the CAR as described herein. In some aspects, the method comprises transfecting or transducing immune effector cells isolated from a subject, such as a subject having a Claudin-1 expressing tumor cell, such that the immune effector cells express one or more CAR as described herein. In some aspects, the immune effector cells are isolated from an individual and genetically modified without further manipulation in vitro. Such cells can then be directly re-administered into the individual. In some aspects, the immune effector cells are first activated and stimulated to proliferate in vitro prior to being genetically modified to express a CAR. In this regard, the immune effector cells may be cultured before or after being genetically modified (i.e., transduced or transfected to express a CAR as described herein).

Prior to in vitro manipulation or genetic modification of the immune effector cells described herein, the source of cells may be obtained from a subject. In some aspects, the immune effector cells for use with the CARs as described herein comprise T cells. T cells can be obtained from a number of sources, including peripheral blood mononuclear cells, bone marrow, lymph nodes tissue, cord blood, thymus issue, tissue from a site of infection, ascites, pleural effusion, spleen tissue, and tumors. In some aspects, T cell can be obtained from a unit of blood collected from the subject using any number of techniques known to the skilled person, such as FICOLL separation. In some aspects, cells from the circulating blood of an individual are obtained by apheresis. The apheresis product typically contains lymphocytes, including T cells, monocytes, granulocyte, B cells, other nucleated white blood cells, red blood cells, and platelets. In some aspects, the cells collected by apheresis may be washed to remove the plasma fraction and to place the cells in an appropriate buffer or media for subsequent processing. In some aspects, the cells are washed with PBS. In some aspects, the washed solution lacks calcium, and may lack magnesium or may lack many, if not all, divalent cations. As would be appreciated by those of ordinary skill in the art, a washing step may be accomplished by methods known to those in the art, such as by using a semi-automated flow through centrifuge. After washing, the cells may be resuspended in a variety of biocompatible buffers or other saline solution with or without buffer. In some aspects, the undesirable components of the apheresis sample may be removed in the cell directly resuspended culture media.

In some aspects, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1b, CD16, HLA-DR, and CD8. Flow cytometry and cell sorting may also be used to isolate cell populations of interest for use in the present invention.

PBMCs may be used directly for genetic modification with the CARs using methods as described herein. In some aspects, after isolation of PBMC, T lymphocytes are further isolated and in some aspects, both cytotoxic and helper T lymphocytes can be sorted into naive, memory. and effector T cell subpopulations either before or after genetic modification and/or expansion. CD8⁺ cells can be obtained by using standard methods. In some aspects, CD8⁺ cells are further sorted into naive, central memory, and effector cells by identifying cell surface antigens that are associated with each of those types of CD8⁺ cells. In some aspects, memory T cells are present in both CD62L+ and CD62L-subsets of CD8⁺ peripheral blood lymphocytes. PBMC are sorted into CD62L-CD8+and CD62L+CD8+ fractions after staining with anti-CD8 and anti-CD62L antibodies. In some aspects, the expression of phenotypic markers of central memory TCM include CD45RO, CD62L, CCR7, CD28, CD3, and CD 127 and are negative for granzyme B. In some aspects, central memory T cells are CD45RO+. CD62L+, CD8+ T cells. In some aspects, effector T cells are negative for CD62L, CCR7, CD28, and CD127, and positive for granzyme B and perforin. In some aspects, naive CD8+T lymphocytes are characterized by the expression of phenotypic markers of naive T cells including CD62L, CCR7, CD28, CD3, CD 127, and CD45RA.

In some aspects, CD4+ T cells are further sorted into subpopulations. For example, CD4+ T helper cells can be sorted into naive, central memory. and effector cells by identifying cell populations that have cell surface antigens. CD4+ lymphocytes can be obtained by standard methods. In some aspects. naive CD4+ T lymphocytes are CD45RO−, CD45RA+, CD62L+CD4+ T cell. In some aspects. central memory CD4+ cells are CD62L positive and CD45RO positive. In some aspects, effector CD4+ cells are CD62L and CD45RO negative.

The immune effector cells, such as T cells, can be genetically modified following isolation using known methods, or the immune effector cells can be activated and expanded (or differentiated in the case of progenitors) in vitro prior to being genetically modified. In some aspects, the immune effector cells, such as T cells, are genetically modified with the chimeric antigen receptors described herein (e.g., transduced with a viral vector comprising a nucleic acid encoding a CAR) and then are activated and expanded in vitro. Methods for activating and expanding T cells are known in the art and are described, for example, in U.S. Pat. Nos. 6,905,874; 6,867,041; 6,797,514; WO2012079000. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). In some aspects, the T cells may be activated and stimulated to proliferate with feeder cells and appropriate antibodies and cytokines using methods such as those described in U.S. Pat. Nos. 6,040,177; 5,827,642; and WO2012129514.

### III. CD44

CD44, which is encoded by CD44 gene on the short arm of chromosome 11 in human, is a type of complex cell-surface glycoprotein (Xu, H., et al., Onco Targets Ther. 8:3783-3792 (2015)). The CD44 gene is composed of 19 exons in human, among which the first five exons (exons 1-5) and the last five exons (exons 16-20) constantly encode CD44s which is the most common and the smallest CD44 protein with a molecule weight of 85-95 kDa (Naor, D., et al., 39:527-579 (2002)). The exons 1-5 and exons 16-20, regarded as stable exons, encode N-terminal containing HA-binding region and C-terminal domain of CD44 protein, respectively (Orian-Rousseau, V., et al., Adv Cancer Res. 123:231-254 (2014)). The middle nine exons can be alternatively spliced and located between exons 1-5 domain and exons 16-20 region, which form multiple different permutations and subsequently encodes manifold CD44v, CD44 consists of three regions, including ectodomain, transmembrane region and intracellular tail (Chen C., et al., J Hematol Oncol. 11:64 (2018); Idzerda, R L, et al., Proc Natl Acad Sci USA 86:4659-4663 (1989)). CD44v isoforms contain an additional stem membraneproximal portion which is encoded by a single variant exon or multifarious conceivable combinations of variant exons. The CD44 peptide can be further processed by glycosylation and addition of heparin sulfate or chondroitin sulfate (Bennett, K L, et al., J Cell Biol 128:687-698 (1995); Greenfield, B., et al., J Biol Chem 274:2511-2517 (1999)).

Cluster of differentiation 44 (CD44) is a complex transmembrane adhesion glycoprotein, and associates with the pivotal component of the extracellular matrix (ECM) hyaluronic acid (HA). See Xu, H., et al., Exp Hematol Oncol 9:36 (2020); see also Thorne. R. F., et al., Histochem Cell Biol. 53:77-87 (2020). CD44 expresses in a variety of cell types in humans, including embryonic stem cells, differentiated cells and cancer cells. See Chen C., et al., J Hematol Oncol. 11:64 (2018). Distinct alternative splicing during the transcription process produces two isoforms of CD44, including the standard isoform (CD44s) and CD44 variant isoforms (CD44v). See Xu, H., et al., Onco Targets Ther. 8:3783-3792 (2015). Numerous studies have reported that CD44 not only prominently participates in normal cellular functions during physiological processes, but also plays pivotal roles in pathological processes, especially tumors. See Domev, H., et al., Tissue Eng Part A 18:2290-2302 (2012); Sneath R J, et al., Mol Pathol 51:191-200 (1998); and Zeilstra J., et al., Oncogene 33:665-670 (2014). CD44 plays important roles in diverse physiological processes, such as organ development, diverse immune functions and haematopoiesis. See Ponta, H., et al., Nat Rev Mol Cell Biol. 4:33-45 (2003). CD44-mediating processes include T cell differentiation, branching morphogenesis, proliferation, adhesion and migration. Id. CD44 as a surface biomarker of cancer stem cells (CSCs) and a vital regulatory factor of epithelial-mesenchymal transition (EMT) program is involved in the regulation of tumor initiation and development See Xu. H., et al., Onco Targets Ther. 8:3783-3792 (2015); Su M., et al., Int J Biol Sci 15:1252-1260 (2019); Skandalis, SS, et al., Cell Signal 63.109377 (2019); and Leng, Z., et al., Cell Physiol Biochem 46:860-872 (2018). Aberrant expression of CD44 and dysregulation of CD44 contribute to tumor formation of multiple cancer entities, including lung cancer (Wang, C Y, et al., J Chin Med Assoc. 82:196-201 (2019)), hepatocellular carcinoma (Asai. R., et al., Cancer Med 8:773-782 (2019)), ovarian cancer (Wang. Y., et al., Oncol Rep. 42:91-102 (2019)), glioma (Hou C., et al., Cancer Res. 145:201-210 (2019)), papillary thyroid carcinoma (Kawai T., et al., Kobe J Med Sci 65: E1-e9 (2019)), head and neck squamous cell carcinoma (HNSCC) (Ludwig, N., et al., Cancer Lett. 467:85-95 (2019)), astrocytic gliomas (Valkonen M., et al., BMC Cancer 18:664 (2018)), and oral squamous cell carcinoma (OSCC) (Shah. K., et al., J Oral Pathol Med 47:345-352 (2018). The 3′ untranslated region of CD44. acting as a competing endogenous RNA to microRNA-34a, boosts the sensitivity of liver CSCs to natural kill cells-mediated cytotoxicity via regulating UL16 binding protein 2 (Weng J., et al., Int J Biol Sci. 15:1664-1675 (2019)). In addition, CD44 also exerts significant effects on caner invasion and metastasis of various tumor types (Chaffer, C L. et al., Dev Cell 47:691-693 (2018)), such as lung adenocarcinoma (Wang, C Y, et al., J Chin Med Assoc. 82:196-201 (2019)), breast cancer (Zhao P., et al., J Cell Sci. 129:1355-1365 (2016); Xu, H., et al., Int J Oncol 49:1343-1350 (2016); and Liu, X., et al., Cancer Discov. 9:96-113 (2019)). neuroblastoma (Vega, F M, et al, EBioMedicine 49:82-95 (2019)), gastric cancer (Shu. X., et al, Mol Cell Biochem 459:35-47 (2019)), esophageal squamous cell carcinoma (ESCC) (Zuo, J., et al., Mol Cell Biochem 443:139-149 (2018)). colorectal cancer (Sadeghi. A., et al., Biomark Med. 13: 419-426 (2019); Mohamed, S. Y., et al., J Gastrointest Cancer 50:824-837 (2019); Chaitra, LP, et al., J Cancer Res Ther. 15:75-81 (2019); and Wang, X F, et al., Zhonghua Yi Xue Za Zhi 98:2899-2904 (2018)), prostate cancer (Senbanjo, LT, et al., Cell Commun Signal 17:80 (2019)), nasopharyngeal carcinoma (Chan, L S, et al., Int J Oncol. 54:1010-1020 (2019)). endometrial cancer (Klaric, M., et al., Appl Immunohistochem Mol Morphol 27:606-612 (2019)), clear cell renal cell (RCC) carcinoma (Zanjani, L S, et al., Biomark Med. 12:45-61 (2018)), pancreatic cancer (Liu, Y., et al., Int J Biol Markers 33:308-313 (2018)), meningioma (Li, H Z, et al., J Biol Regul Homeost Agents 32:69-74 (2018)) and ovarian cancer (Bhattacharya, R., et al., J Cell Biochem 119:3373-3383 (2018)). As has been reported, the intracellular domain (ICD) of CD44 interacting with RUNX2 to form a co-transcription factor drives the migration of prostate cancer cell line PC3 through upregulating the levels of metastasis-related genes, such as matrix metalloproteinase 9 (MMP9) and osteopontin (Senbanjo, L T, et al., Cell Commun Signal 17:80 (2019)).

In some aspects, the CD44 protein is encoded by the nucleotide sequence corresponding to NCBI Ref. Seq. Accession Number NM_000610.4 (SEQ ID NO: 15). In some aspects, the CD44 protein comprises the amino acid sequence corresponding to NCBI Ref. Seq. Accession Number NP_000601.3 (SEQ ID NO: 16).

CD44 has previously been used for predicting the prognosis of cancer patients (Shu, C., et al., Clin Transl Oncol. 20:1439-1447 (2018); Zeng, L., et al., Medicine 99: e20428 (2020); Sawant, S., et al., J Oral Pathol Med. 47:665-673 (2018); Karan, Križanac, et al., Appl Immnuohistochem Mol Morphol. 26:398-402 (2018); Huang, H H, et al., PLOS ONE 13.e0206685 (2018); Bitaraf, S M, et al., Asian Pac J Cancer Prev. 19:1313-1318 (2018); and Sun. H., et al., Genes Chromosomes Cancer 56:598-607 (2017). For example, based on immunohistochemistry analysis of 125 breast cancer patient samples, it was found that CD44 protein level was positively correlated with poor disease-free survival (DFS) and OS (Xie. X., et al., Curr Cancer Drug Targets 18:592-599 (2018)). Additionally. in another study, to immunohistochemistry analysis of a total of 206 RCC samples, high protein abundance of CD44 was correlated with malignant phenotype and unfavorable clinical outcomes of clear cell RCC rather than another two RCC subtypes (papillary and chromophobe RCC) (Zanjani, L S, et al., Biomark Med. 12:45-61 (2018)). A correlation between CD44 and Claudin-1 has previously been shown in ovarian cancer as well. (Visco, Z., et al., Front. Oncol. 11:620873, 15 pages). Additionally, a relationship between CD44, CLDN1, and NF-κB has been highlighted in the tumor microenvironment (Guo, J Y., et al., Oncogene. 36:2457-2471 (2017), doi.org/10.1038/onc.2016.404)

In some aspects, CD44 can be used as a biomarker to measure anti-Claudin-1-antibody target engagement in a fibrosis. In some aspects, the fibrosis is kidney fibrosis, lung fibrosis, or liver fibrosis. In some aspects, the fibrosis is kidney fibrosis.

In some aspects, soluble CD44 levels are measured by an enzyme-linked immunoassay (ELISA). In some aspects, soluble CD44 levels are measured by mass spectrometry.

### IV. CD147

Cluster of Differentiation (CD147) is a glycoprotein initially known as a regulator of MMPs. through cell-matrix and cell-cell interaction (Landras. A., et al, Cancers 11:1803 (2019)). CD147 is coded by Basigin (BSG) gene located on chromosome 19p13.3 (Kaname, T., et al., Cell Genet. 64.195-197 (1993)). Four variants of CD147 have been encoded (Basigin 1, 2, 3 and 4) through an alternative promoter and splicing (Liao, C. -G., et al., Mol. Cell. Biol. 31:2591-2604 (2011). The retinal-specific CD147 (Basigin 1) containing three immunoglobulin domains (Hanna, S. M., et al., BMC Biochem 4:17 (2003)) and isoforms Basigin 3 and 4 contain a single immunoglobulin domain (Belton, R. J., et al., J. Biol. Chem. 283:17805-17814 (2008)). The most abundant isoform and complete information is only available for Basigin 2, named CD147 in the present review. It is composed of a signal sequence of 21 amino acids, an extracellular domain of 184 amino acids with two immunoglobulin domains, a transmembrane domain of 24 amino acids. and a cytoplasmic domain of 39 amino acids (Muramatsu, T., et al., Histol. Histopathol. 18:981-987 (2003)). Structurally. one monomer of CD147 is composed of two domains, D1 corresponding to N-terminal domain (residues 22-101) and a C-terminal domain called D2 (residues 107-205). Crystal structure showed CD147 monomers can associate with each other, leading to dimer formation (Yu. X. -L., et al., J. Biol. Chem. 283 18056-18065 (2008)). It was first shown that CD147 dimerization can occur in the same cell (Fadool, J. M., et al., Biochem. Biophys. Res. Commun. 229:280-286 (1996)). This homodimerization on the plasma membrane is in a cis-dependent manner (Yoshida, S., et al., Eur. J. Biochem 267:4372-4380 (2000)). CD147 can also interact with other cells through CD147/CD147 interaction in trans-dependent manner, which can induce intracellular signaling (Schmidt, R., et al., Circ. Res. 102:302-309 (2008)). Moreover, a soluble form of CD147 was shown to be internalized through surface CD147 binding and enhance proliferation and migration (Knutti, N., et al., FEBS J. 282:4187-4200 (2015)). This interaction can then induce cell surface expression of CD 147. Id. Dimerization of CD 147 is crucial for MMPs induction and cell invasion in hepatocellular carcinoma though MAPK pathway (Cui. H. -Y., et al., Biochem. Biophys. Res. Commun. 419:517-522 (2012)). Cancer-associated fibroblasts (CAFs) are the most abundant components of tumoral stroma and contribute to the malignant phenotype of cancers. In 2019, Aoki et al. demonstrated that CD147 can stimulate adjacent fibroblasts thought CD73 interaction, increasing the secretion of MMP-2 and promoting invasion and metastasis (Aoki, M., et al., BMC Cancer 19:1-13 (2019)). CD 147 has been shown to be implicated in the transformation of normal fibroblasts to cancer-associated-fibroblast through cancer-stroma interaction and the induction of alpha smooth muscle actin (α-SMA) expression, a marker of CAFs, promoting epithelial-to-mesenchymal transition of breast cancer cells (Xu, J., et al., Cancer Lett. 335:380-386 (2013)). CD147 contains three asparagine (Asn) glycosylation sites in the extracellular region. The glycosylation level of CD147 is related to its different molecular weights, which have been described and classified as low and high glycosylated CD147 forms (Yoshida, S., et al., Eur. J. Biochem 267:4372-4380 (2000)). The glycosylation of CD147 was shown to be necessary for its function, since the unglycosylated forms of CD147 were not able to induce fibroblast's MMPs production (Tang, W., et al., Mol. Biol. Cell, 15:4043-4050 (2004)).

An important mechanism of action of CD147 is that it can be secreted by cells and released into the microenvironment. Two different ways by which the cell can generate these soluble forms of CD147 have been described; one involving an MTI-MMP-mediated cleavage of surface-bound CD147 (Egawa, N., et al., J. Biol. Chem. 281:37576-37585 (2006)) and a second is based on the release of microvesicles, containing full-length of CD147 molecules (Sidhu. S. S., et al., Oncogene 23:956 (2004)) Recently, the interaction between ADAM12 and CD147 was shown to promote the cleavage of CD147 releasing its soluble form in the microenvironment, showing a new way to generate soluble CD147 (Albrechtsen, R., et al., Int. J. Mol. Sci. 20:1957 (2019)). A soluble form of CD147 was reported to be associated with tumor growth, metastasis formation and chemoresistance, and has been proposed as a clinical biomarker in breast cancer (Kuang. Y. H., et al., Hong Kong Med. J. 24:252-260 (2018)) and hepatocellular carcinoma (Lee, A., et al., J. Gastroenterol. Hepatol. 31:459-466 (2016)).

In some aspects, the CD147 protein is encoded by the nucleotide sequence corresponding to NCBI Ref. Seq. Accession Number NM_001322243.2 (SEQ ID NO: 17). In some aspects, the CD147 protein comprises the amino acid sequence corresponding to NCBI Ref. Seq. Accession Number NP_001309172.1 (SEQ ID NO: 18).

In some aspects, CD 147 can be used as a biomarker to measure anti-Claudin-1-antibody target engagement in a fibrosis. In some aspects, the fibrosis is kidney fibrosis, lung fibrosis, or liver fibrosis. In some aspects, the fibrosis is kidney fibrosis.

In some aspects, soluble CD 147 levels are measured by an enzyme-linked immunoassay (ELISA). In some aspects, soluble CD147 levels are measured by mass spectrometry.

### V. Osteopontin (SPP1)

Osteopontin, or secreted phosphoprotein 1 (SPP1), refers to a family of phosphorylated glycoproteins that are abundant in bone mineral matrix and accelerates bone regeneration and remodeling. It is also produced in other tissues and plays a role in the regulation and progression of many diseases, such as fibrosis. Exemplary accession numbers for representative nucleotide and protein sequences for osteopontin include but are not limited to: AAA62729, AAA59974, CAA 40091, AAC28619, NM_000582.3, NM_001040058.2, NM_001040060.2, NM_001251829.2, NM_001251830.2, NP_000573.1, NP_001035147.1, NP_001035149.1, NP 001238758.1 and NP 001238759.1.

In some aspects, the SPP1 protein is encoded by the nucleotide sequence corresponding to NCBI Ref Seq. Accession Number NM_000582.3 (SEQ ID NO: 26). In some aspects, the SPP1 protein comprises the amino acid sequence corresponding to NCBI Ref. Seq. Accession Number NP_000573.1 (SEQ ID NO: 27).

In some aspects, SPP1 can be used as a biomarker to measure anti-Claudin-1-antibody target engagement in a fibrosis. In some aspects, the fibrosis is kidney fibrosis, lung fibrosis, or liver fibrosis. In some aspects, the fibrosis is kidney fibrosis.

In some aspects, soluble SPP1 levels are measured by an enzyme-linked immunoassay (ELISA). In some aspects, soluble SPP1 levels are measured by mass spectrometry.

### VI. Detection Methods

In certain aspects, identifying a patient suitable for an anti-Claudin-1 antibody therapy for the present methods includes measuring or assessing a CD44, CD147 and/or SPP1 expression in a sample, for example, a fibrotic test tissue sample. The methods of measuring or assessing the CD44, CD147 and/or SPP1 expression can be achieved by any methods applicable.

In order to assess the CD44, CD147 and/or SPP1 expression, in one aspect, a test tissue sample is obtained from the patient who is in need of the therapy. In some aspects, a test tissue sample includes, but is not limited to, any clinically relevant tissue sample, such as a tumor biopsy, a core biopsy tissue sample, a fine needle aspirate, or a sample of bodily fluid, such as blood, plasma, serum, lymph, ascites fluid, cystic fluid, or urine. In some aspects, the test tissue sample is from a primary tumor. In some aspects, the test tissue sample is from a metastasis. In some aspects, test tissue samples are taken from a subject at multiple time points, for example, before treatment, during treatment, and/or after treatment. In some aspects, test tissue samples are taken from different locations in the subject, for example, a sample from a primary tumor and a sample from a metastasis in a distant location. In some aspects, the test tissue sample is from a fibrotic tissue sample.

In some aspects, the test tissue sample is a paraffin-embedded fixed tissue sample. In some aspects, the test tissue sample is a formalin-fixed paraffin embedded (FFPE) tissue sample. In some aspects, the test tissue sample is a fresh tissue (e.g., tumor) sample. In some aspects, the test tissue sample is a frozen tissue sample. In some aspects, the test tissue sample is a fresh frozen (FF) tissue (e.g., fibrotic) sample. In some aspects, the test tissue sample is a cell isolated from a fluid. In some aspects, the test tissue sample comprises circulating tumor cells (CTCs) In some aspects, the test tissue sample comprises tumor-infiltrating lymphocytes (TILs). In some aspects, the test tissue sample comprises tumor cells and tumor-infiltrating lymphocytes (TILs). In some aspects. the test tissue sample comprises circulating lymphocytes. In some aspects, the test tissue sample is an archival tissue sample. In some aspects, the test tissue sample is an archival tissue sample with known diagnosis, treatment, and/or outcome history. In some aspects, the sample is a block of tissue. In some aspects, the test tissue sample is dispersed cells. In some aspects, the sample size is from about 1 cell to about 1×10⁶ cells or more. In some aspects, the sample size is about 1 cell to about 1×10⁵ cells. In some aspects, the sample size is about 1 cell to about 10,000 cells. In some aspects, the sample size is about 1 cell to about 1,000 cells. In some aspects, the sample size is about 1 cells to about 100 cells. In some aspects, the sample size is about 1 cell to about 10 cells. In some aspects, the sample size is a single cell.

In some aspects, the test tissue sample is obtained from a liquid biopsy. In some aspects, the test tissue sample is urine, plasma, or serum. In some aspects, the test tissue sample is obtained by a nasal brush. In some aspects, the test tissue sample is obtained by a hair biopsy or scalp biopsy. In some aspects, the test tissue sample is obtained by bronchoalveolar lavage.

In another aspects, the assessment of CD44, CD147 and/or SPP1 expression can be achieved without obtaining a test tissue sample. In some aspects, selecting a suitable patient includes (i) optionally providing a test tissue sample obtained from a patient with fibrosis of the tissue, the test tissue sample comprising fibrotic cells; and (ii) assessing the proportion of cells in the test tissue sample that express CD44, CD147 and/or SPP1 on the surface of the cells based on an assessment that the proportion of cells in the test tissue sample that express CD44, CD147 and/or SPP1 on the cell surface is higher than a predetermined threshold level.

In any of the methods comprising the measurement of CD44, CD147 and/or SPP1 expression in a test tissue sample, however, it should be understood that the step comprising the provision of a test tissue sample obtained from a patient is an optional step. That is, in certain aspects, the method includes this step, and in other aspects, this step is not included in the method. It should also be understood that in certain aspects the “measuring” or “assessing” step to identify, or determine the number or proportion of, cells in the test tissue sample that express CD44, CD147 and/or SPP1 is performed by a transformative method of assaying for CD44, CD147 and/or SPP1 expression, for example by performing a reverse transcriptase-polymerase chain reaction (RT-PCR) assay or an IHC assay. In certain other aspects, no transformative step is involved and CD44, CD147 and/or SPP1 expression is assessed by, for example, reviewing a report of test results from a laboratory. In some aspects, CD44, CD147 and/or SPP1 expression is assessed by reviewing the results of an immunohistochemistry assay from a laboratory. In certain aspects, the steps of the methods up to, and including, assessing CD44, CD147 and/or SPP1 expression provides an intermediate result that may be provided to a physician or other healthcare provider for use in selecting a suitable candidate for the combination therapy of an anti-Claudin-1 antibody therapy. In certain aspects, the steps of the methods up to, and including, assessing CD44, CD147 and/or SPP1 expression provides an intermediate result that may be provided to a physician or other healthcare provider for use in selecting a suitable candidate for treatment. In certain aspects, the steps of the methods up to, and including, assessing CD44, CD147 and/or SPP1 expression provides an intermediate result that may be provided to a physician or other healthcare provider for use in selecting a suitable candidate for anti-Claudin-1 antibody therapy. In certain aspects, the steps that provide the intermediate result is performed by a medical practitioner or someone acting under the direction of a medical practitioner. In other aspects, these steps are performed by an independent laboratory or by an independent person such as a laboratory technician.

In certain aspects of any of the present methods, the proportion of cells that express CD44, CD147 and/or SPP1 is assessed by performing an assay to detect the presence of CD44, CD147 and/or SPP1 RNA. In further aspects, the presence of CD44, CD147 and/or SPP1 RNA is detected by RT-PCR, in situ hybridization or RNase protection. In some aspects, the presence of CD44, CD147 and/or SPP1 RNA is detected by an RT-PCR based assay. In some aspects, scoring the RT-PCR based assay comprises assessing the level of CD44, CD147 and/or SPP1 RNA expression in the test tissue sample relative to a predetermined level.

In other aspects, the amount of soluble CD44, CD147 and/or SPP1 is assessed by performing an assay to detect the presence of CD44, CD147 and/or SPP1 polypeptide. In further aspects, the presence of CD44, CD147 and/or SPP1 polypeptide is detected by IHC, enzyme-linked immunosorbent assay (ELISA), in vivo imaging, or flow cytometry. In some aspects, CD44, CD147 and/or SPP1 expression is assayed by IHC. In other aspects of all of these methods, cell surface expression of CD44, CD147 and/or SPP1 is assayed using, e.g., IHC or in vivo imaging.

In certain aspects of any of the present methods, the proportion of cells that express CD44, CD147 and/or SPP1 in the test tissue sample is assessed by performing an assay to detect the presence of CD44, CD147 and/or SPP1 polypeptide. In some aspects, the presence of CD44, CD147 and/or SPP1 polypeptide is detected by an immunohistochemistry assay. In some aspects, the test tissue sample is a tumor biopsy. In some aspects, the test tissue sample is a formalin-fixed paraffin embedded (FFPE) sample.

In some aspects, the immunohistochemistry assay is a monoplex assay. In some aspects, the immunohistochemistry assay is a multiplex assay.

In one aspects of the present methods, an automated IHC method is used to assay the expression of CD44, CD147 and/or SPP1 in FFPE tissue specimens. This disclosure provides methods for detecting the presence of human CD44, CD147 and/or SPP1 antigen in a test tissue sample, or quantifying the level of human CD44, CD 147 and/or SPP1 antigen or the proportion of cells in the sample that express the antigen, which methods comprise contacting the test sample, and a negative control sample, with an antibody that specifically binds to human CD44 or CD147, under conditions that allow for formation of a complex between the antibody or portion thereof and human CD44, CD147 and/or SPP1 In certain aspects, the test and control tissue samples are FFPE samples. The formation of a complex is then detected, wherein a difference in complex formation between the test sample and the negative control sample is indicative of the presence of human CD44. CD147 and/or SPP1 antigen in the sample. Various methods are used to quantify CD44, CD 147 and/or SPP1 expression.

In a particular aspects, the automated IHC method comprises: (a) deparaffinizing and rehydrating mounted tissue sections in an autostainer; (b) retrieving antigen in an autostainer; (c) setting up reagents on an autostainer; and (d) running the autostainer to include steps of neutralizing endogenous peroxidase in the tissue specimen; blocking non-specific protein-binding sites on the slides; incubating the slides with primary Ab, incubating with a postprimary blocking agent; incubating with a postprimary antibody detection agent, such as another antibody that may or may not be conjugated to a detection enzyme; incubating with a polymeric-enzyme detection reagent; adding a chromogen substrate and developing; and counterstaining with hematoxylin. In some aspects, the retrieving antigen comprises using any heat based antigen retrieval device.

### VII. Treatment Methods

The disclosure provides a method for identifying a subject having fibrosis that is suitable for therapy with an anti-Claudin-1 antibody or antigen binding fragment thereof, comprising the steps of: a) obtaining a biological sample a human subject; b) detecting the expression levels of one or more biomarkers selected from the group consisting of CD44, CD147, osteopontin (SPP1) and a combination thereof; c) comparing the detected expression level of the one or more biomarkers with a control level of the one or more biomarker; and d) identifying the human subject as a responder when the detected expression level of the one or more biomarkers is greater than the control level of CD44, CD147, or SPP1 expression, thereby identifying the human subject having fibrosis that is suitable for therapy with the anti-Claudin-1 antibody or antigen binding fragment thereof. In some aspects, the method further comprises: e) administering the anti-Claudin-1 antibody or antigen binding fragment thereof in an amount sufficient to alleviate a symptom of fibrosis when the subject is identified as a responder.

The disclosure provides a method of identifying fibrosis progression in a human subject that is suitable for therapy with an anti-Claudin-1 antibody or an antigen binding fragment thereof comprising the steps of: a) obtaining a biological sample a human subject; b) detecting the expression levels of one or more biomarkers selected from the group consisting of CD44, CD147, SPP1 and a combination thereof; c) comparing the detected expression level of the one or more biomarkers with a control level of the one or more biomarker; and d) identifying the human subject as a responder when the detected expression level of the one or more biomarkers is greater than the control level of CD44, CD147, or SPP1 expression, thereby identifying fibrosis progression in the human subject that is suitable for therapy with an anti-Claudin-1 antibody or an antigen binding fragment thereof. In some aspects, the method further comprises: e) administering the anti-Claudin-1 antibody or antigen binding fragment thereof in an amount sufficient to alleviate a symptom of fibrosis progression when the subject is identified as a responder.

The disclosure provides a method for treating a subject having fibrosis, the method comprising the steps of: a) obtaining a biological sample a human subject; b) detecting the expression levels of one or more biomarkers selected from the group consisting of CD44, CD147, SPP1 and a combination thereof; c) comparing the detected expression level of the one or more biomarkers with a control level of the one or more biomarker; and d) identifying the human subject as a responder when the detected expression level of the one or more biomarkers is greater than the control level of CD44, CD147, or SPP1 expression, and e) administering the anti-Claudin-1 antibody or antigen binding fragment thereof in an amount sufficient to alleviate a symptom of fibrosis when the subject is identified as a responder, thereby treating the subject having fibrosis.

In some aspects, provided herein is a method of treating fibrosis comprising: (a) administering an anti-Claudin-1 antibody to a subject; (b) determining a level of soluble CD44 in a test sample from the subject; and (c) comparing the level of soluble CD44 in the test sample to a level of soluble CD44 in a control sample. In some aspects, if the level of soluble CD44 in the test sample is not increased relative to level of soluble CD44 in the control sample, then the dose of the anti-Claudin-1 antibody being administered to the subject is increased. In some aspects, if the level of soluble CD44 in the test sample is increased relative to the control sample, then the dose of the anti-Claudin-1 antibody being administered to the subject is not increased.

In some aspects, if the level of soluble CD44 in the test sample is not increased relative to level of soluble CD44 in the control sample, then the subject is administered nintedanib or pirfenidone.

In some aspects, provided herein is a method of treating fibrosis in a subject. In some aspects, the subject has been determined to have a level of soluble CD44 in a test sample from the subject that is not increased relative to a level of soluble CD44 in a control sample. In some aspects, the subject has previously been administered an anti-Claudin-1 treatment, the method comprising administering an increased dose of the anti-Claudin-1 treatment to the subject.

In some aspects, provided herein is a method of monitoring fibrosis progression in a subject comprising: (a) quantifying a level of soluble CD44 in a test sample from the subject, wherein the subject has previously been administered an anti-Claudin-1 treatment; and (b) comparing the level of soluble CD44 in the test sample to a level of soluble CD44 in a control sample. In some aspects, a level of soluble CD44 in the test sample that is not increased relative to the level of soluble CD44 in the control sample indicates that the subject is susceptible to an increased dose of the anti-Claudin-1 treatment.

In some aspects, provided herein is a method of monitoring fibrosis progression in a subject comprising: (a) determining a level of soluble CD44 in a test sample from the subject; and (b) comparing the level of soluble CD44 in the test sample to a level of soluble CD44 in a control sample. In some aspects, the subject has previously been administered an anti-Claudin-1 antibody. In some aspects, a level of soluble CD44 in the test sample that is not increased relative to the level of soluble CD44 in the control sample indicates that the subject is susceptible to an increased dose of the anti-Claudin-1 antibody.

In some aspects, provided herein is a method for treating a subject suffering from fibrosis, the method comprising the steps of: determining whether the subject has increased levels of soluble CD44 by obtaining a test sample from the subject and comparing the level of soluble CD44 in the test sample to a control sample. In some aspects, the subject has previously been administered a dose of an anti-Claudin-1 antibody. In some aspects, if the subject has increased levels of soluble CD44 in the test sample relative to the control sample, then the subject is not administered an increased dose of the anti-Claudin-1 antibody. In some aspects, if the subject does not have increased levels of soluble CD44 in the test sample relative to the control sample. then the subject is administered an increased dose of the an anti-Claudin-1 antibody.

In some aspects, provided herein is a method for designing a customized therapy for a subject suffering from fibrosis which comprises (a) quantifying the level of soluble CD44 in a test sample from the subject; (b) comparing the level of soluble CD44 in the test sample obtained in step (a) with the level of soluble CD44 in a control sample; (c) determining that the subject has a not increased level of soluble CD44 with respect to the level of soluble CD44 in the control sample; and (d) administering an increased dose of the anti-Claudin-1 antibody. In some aspects, the subject has previously been administered a dose of an anti-Claudin-1 antibody. In some aspects, if the level of soluble CD44 in the test sample obtained in step (a) is not increased relative to the level of soluble CD44 in the control sample, then the subject is susceptible to receive an increased dose of the anti-Claudin-1 antibody.

In some aspects, provided herein is a method of classifying a subject suffering from fibrosis into a cohort and treating said subject, comprising (a) quantifying the level of soluble CD44 in a test sample from the subject; (b) comparing the level of soluble CD44 in the test sample obtained in step (a) with the level of soluble CD44 in a control sample; (c) determining that the subject has a not increased level of soluble CD44 with respect to the level of soluble CD44 in the control sample; (d) classifying said subject into a cohort based on the level of soluble CD44 in the test sample relative to the control sample; and (c) administering an increased dose of the anti-Claudin-1 antibody. In some aspects, the subject has previously been administered a dose of an anti-Claudin-1 antibody. In some aspects, if the level of soluble CD44 in the test sample obtained in step (a) is not increased relative to the level of soluble CD44 in the control sample, then the subject is susceptible to receive an increased dose of the anti-Claudin-1 antibody.

In some aspects, provided herein is a method of treating fibrosis comprising: (a) administering an anti-Claudin-1 antibody to a subject; (b) determining a level of soluble CD147 in a test sample from the subject; and (c) comparing the level of soluble CD 147 in the test sample to a level of soluble CD147 in a control sample. In some aspects, if the level of soluble CD147 in the test sample is not increased relative to level of soluble CD147 in the control sample, then the dose of the anti-Claudin-1 antibody being administered to the subject is increased. In some aspects, if the level of soluble CD147 in the test sample is increased relative to the control sample, then the dose of the anti-Claudin-1 antibody being administered to the subject is not increased.

In some aspects, if the level of soluble CD 147 in the test sample is not increased relative to level of soluble CD 147 in the control sample, then the subject is administered nintedanib or pirfenidone.

In some aspects, provided herein is a method of treating fibrosis in a subject that has been determined to have a level of soluble CD147 in a test sample from the subject that is not increased relative to a level of soluble CD 147 in a control sample. In some aspects, the subject has previously been administered an anti-Claudin-1 treatment. In some aspects, the method comprises administering an increased dose of the anti-Claudin-1 treatment to the subject.

In some aspects, provided herein is a method of monitoring fibrosis progression in a subject comprising: (a) quantifying a level of soluble CD147 in a test sample from the subject; and (b) comparing the level of soluble CD147 in the test sample to a level of soluble CD147 in a control sample. In some aspects, the subject has previously been administered an anti-Claudin-1 treatment. In some aspects, a level of soluble CD 147 in the test sample that is not increased relative to the level of soluble CD147 in the control sample indicates that the subject is susceptible to an increased dose of the anti-Claudin-1 treatment.

In some aspects, provided herein is a method of monitoring fibrosis progression in a subject comprising: (a) determining a level of soluble CD147 in a test sample from the subject; and (b) comparing the level of soluble CD147 in the test sample to a level of soluble CD147 in a control sample. In some aspects, the subject has previously been administered an anti-Claudin-1 antibody. In some aspects, a level of soluble CD147 in the test sample that is not increased relative to the level of soluble CD147 in the control sample indicates that the subject is susceptible to an increased dose of the anti-Claudin-1 antibody.

In some aspects, provided herein is a method for treating a subject suffering from fibrosis, the method comprising the steps of: determining whether the subject has increased levels of soluble CD147 by obtaining a test sample from the subject and comparing the level of soluble CD147 in the test sample to a control sample. In some aspects, the subject has previously been administered a dose of an anti-Claudin-1 antibody. In some aspects, if the subject has increased levels of soluble CD147 in the test sample relative to the control sample, then the subject is not administered an increased dose of the anti-Claudin-1 antibody. In some aspects, if the subject does not have increased levels of soluble CD 147 in the test sample relative to the control sample, then the subject is administered an increased dose of an anti-Claudin-1 antibody.

In some aspects, provided herein is a method for designing a customized therapy for a subject suffering from fibrosis which comprises (a) quantifying the level of soluble CD147 in a test sample from the subject; (b) comparing the level of soluble CD147 in the test sample obtained in step (a) with the level of soluble CD147 in a control sample; (c) determining that the subject has a not increased level of soluble CD147 with respect to the level of soluble CD147 in the control sample; and (d) administering an increased dose of the anti-Claudin-1 antibody. In some aspects, the subject has previously been administered a dose of an anti-Claudin-1 antibody. In some aspects, if the level of soluble CD147 in the test sample obtained in step (a) is not increased relative to the level of soluble CD 147 in the control sample, then the subject is susceptible to receive an increased dose of the anti-Claudin-1 antibody

In some aspects, provided herein is a method of classifying a subject suffering from fibrosis into a cohort and treating said subject, comprising (a) quantifying the level of soluble CD147 in a test sample from the subject; (b) comparing the level of soluble CD147 in the test sample obtained in step (a) with the level of soluble CD147 in a control sample; (c) determining that the subject has a not increased level of soluble CD147 with respect to the level of soluble CD147 in the control sample; (d) classifying said subject into a cohort based on the level of soluble CD147 in the test sample relative to the control sample; and (e) administering an increased dose of the anti-Claudin-1 antibody. In some aspects, the subject has previously been administered a dose of an anti-Claudin-1 antibody. In some aspects, if the level of soluble CD147 in the test sample obtained in step (a) is not increased relative to the level of soluble CD147 in the control sample, then the subject is susceptible to receive an increased dose of the anti-Claudin-1 antibody.

In some aspects, the subject has previously been administered with anti-Claudin-1 antibody or antigen binding fragment thereof.

In some aspects, the previous administration of anti-Claudin-1 antibody or antigen binding fragment thereof in the subject is administered with a first dose, and when the subject is identified as a responder, the subject is administered with at least a second dose of anti-Claudin-1 antibody or antigen binding fragment thereof.

In some aspects, the first dose and the second dose are equal. In some aspects, the second dose is greater than the first dose.

As a general proposition, the therapeutically effective amount of the anti-Claudin-1 antibody or antigen binding fragment thereof administered to human subject will be in the range of about 0.01 to about 100 mg/kg of patient body weight whether by one or more administrations.

In some aspects, the antibody used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg kg, about 0.01 to about 10 mg/kg, about 0.01 to about 5 mg/kg, or about 0.01 to about 1 mg/kg administered daily, for example.

In some aspects, the anti-Claudin-1 antibody or antigen binding fragment thereof is administered to a human subject at a dose of about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, or about 100 mg/kg.

In some aspects, the anti-Claudin-1 antibody or antigen binding fragment thereof is administered to a human subject at a dose of about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, about 1100 mg/kg, about 1200 mg/kg, about 1300 mg/kg, about 1400 mg/kg or about 1500 mg/kg.

The dose may be administered as a single dose or as multiple doses (e.g., 2 or 3 doses), such as infusions. For repeated administrations over several days or longer, depending on the condition, the treatment would generally be sustained until a desired suppression of disease symptoms occurs. An exemplary dose would be in the range from about 0.3 mg/kg to about 20 mg/kg. Thus, one or more doses of about 0.3 mg/kg, 3 mg/kg, 10 mg/kg, 15 mg/kg or 20 mg/kg (or any combination thereof) may be administered to the patient. Such doses may be administered intermittently. for example, every week or every three weeks (e.g., such that the patient receives from about two to about twenty, or, for example, about three doses of the anti-Claudin-1 antibody or antigen binding fragment thereof). An initial higher loading dose, followed by one or more lower doses may be administered. The progress of this therapy is easily monitored by the methods described herein or conventional techniques and assays.

In some aspects, the dose of the anti-Claudin-1 antibody prior to being increased is 0.3 mg/kg or 3 mg/kg.

In some aspects, the dose of the anti-Claudin-1 antibody after being increased is 3 mg/kg. In some aspects, the dose of the anti-Claudin-1 antibody after being increased is 10 mg/kg. In some aspects, the dose of the anti-Claudin-1 antibody after being increased is 15 mg/kg. In some aspects. the dose of the anti-Claudin-1 antibody after being increased is 20 mg/kg.

In some aspects, the dose of the anti-Claudin-1 antibody prior to being increased is about 0.3 mg/kg, about 3 mg/kg, about 10 mg/kg, about 15 mg/kg or about 20 mg/kg. In some aspects, the dose of the anti-Claudin-1 antibody prior to being increased is at least about 0.3 mg/kg, at least about 3 mg/kg, at least about 10 mg/kg, at least about 15 mg/kg or at least about 20 mg/kg.

In some aspects the first dose is about 0.3 mg/kg, about 3 mg/kg, about 10 mg/kg, about 15 mg/kg or about 20 mg/kg. In some aspects, the first dose administered one time, two times. three times, four times or five times.

In some aspects, the second dose is about 0.3 mg/kg, 3 mg/kg, 10 mg/kg, 15 mg/kg or 20 mg/kg. In some aspects the the second dose is administered one time, two times, three times, four times or five times.

In some aspects, the anti-Claudin-1 antibody is administered intratumorally, intravenously, intraperitoneally, intramuscularly, intrathecally or subcutaneously.

In some aspects, the fibrosis is kidney fibrosis, lung fibrosis, or liver fibrosis. In some aspects, the fibrosis is kidney fibrosis.

In some aspects, the control sample is a sample from the subject prior to being administered an anti-Claudin-1 antibody or an anti-Claudin-1 treatment.

In some aspects, if the subject is susceptible to an increased dose of the anti-Claudin-1 antibody, then the anti-Claudin-1 antibody is administered to the subject. In some aspects, the anti-Claudin-1 treatment is an anti-Claudin-1 antibody.

In some aspects, the level of CD44 in the test sample or the biological sample obtained in step (a) is decreased by at least about 20% relative to the level of CD44 in the control sample. In some aspects, the level of CD44 in the test sample or the biological sample obtained in step (a) is decreased by at least about 5%, at least about 10%, at least about 15% or at least about 20% relative to the level of CD44 in the control sample.

In some aspects, the level of CD44 in the test sample or the biological sample obtained in step (a) is decreased by at least 20% relative to the level of CD44 in the control sample. In some aspects, the level of CD44 in the test sample or the biological sample obtained in step (a) is decreased by at least 5%, at least 10%, at least 15% or at least 20% relative to the level of CD44 in the control sample.

In some aspects, the level of CD44 in the test sample or the biological sample obtained in step (a) is decreased by about 20% relative to the level of CD44 in the control sample. In some aspects, the level of CD44 in the test sample or the biological sample obtained in step (a) is decreased by about 5%, about 10%, about 15% or about 20% relative to the level of CD44 in the control sample. In some aspects, the level of CD44 in the test sample or the biological sample obtained in step (a) is decreased by 20% relative to the level of CD44 in the control sample. In some aspects, the level of CD44 in the test sample or the biological sample obtained in step (a) is decreased by 5%, 10%, 15% or 20% relative to the level of CD44 in the control sample.

In some aspects, the level of CD147 in the test sample or the biological sample obtained in step (a) is decreased by at least about 20% relative to the level of CD147 in the control sample. In some aspects, the level of CD147 in the test sample or the biological sample obtained in step (a) is decreased by at least about 5%, at least about 10%, at least about 15% or at least about 20% relative to the level of CD147 in the control sample.

In some aspects, the level of CD147 in the test sample or the biological sample obtained in step (a) is decreased by at least 20% relative to the level of CD147 in the control sample. In some aspects, the level of CD147 in the test sample or the biological sample obtained in step (a) is decreased by at least 5%, at least 10%, at least 15% or at least 20% relative to the level of CD 147 in the control sample.

In some aspects, the level of CD 147 in the test sample or the biological sample obtained in step (a) is decreased by about 20% relative to the level of CD147 in the control sample. In some aspects, the level of CD147 in the test sample or the biological sample obtained in step (a) is decreased by about 5%, about 10%, about 15% or about 20% relative to the level of CD147 in the control sample. In some aspects, the level of CD147 in the test sample or the biological sample obtained in step (a) is decreased by 20% relative to the level of CD147 in the control sample. In some aspects, the level of CD147 in the test sample or the biological sample obtained in step (a) is decreased by 5%, 10%, 15% or 20% relative to the level of CD147 in the control sample.

In some aspects, the level of SPP1 in the test sample or the biological sample obtained in step (a) is decreased by at least about 20% relative to the level of SPP1 in the control sample. In some aspects, the level of SPP1 in the test sample or the biological sample obtained in step (a) is decreased by at least about 5%, at least about 10%, at least about 15% or at least about 20% relative to the level of SPP1 in the control sample.

In some aspects, the level of SPP1 in the test sample or the biological sample obtained in step (a) is decreased by at least 20% relative to the level of SPP1 in the control sample. In some aspects, the level of SPP1 in the test sample or the biological sample obtained in step (a) is decreased by at least 5%, at least 10%, at least 15% or at least 20% relative to the level of SPP1 in the control sample.

In some aspects, the level of SPP1 in the test sample or the biological sample obtained in step (a) is decreased by about 20% relative to the level of SPP1 in the control sample. In some aspects, the level of SPP1 in the test sample or the biological sample obtained in step (a) is decreased by about 5%, about 10%, about 15% or about 20% relative to the level of SPP1 in the control sample. In some aspects, the level of SPP1 in the test sample or the biological sample obtained in step (a) is decreased by 20% relative to the level of SPP1 in the control sample. In some aspects, the level of SPP1 in the test sample or the biological sample obtained in step (a) is decreased by 5%, 10%, 15% or 20% relative to the level of SPP1 in the control sample.

In some aspects, the level of CD44 in the control sample is about 50 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1050 ng/mL, about 1100 ng/m, about 1150 ng/mL, about 1200 ng/mL, about 1250 ng/mL, about 1300 ng/mL, about 1350 ng/mL, about 1400 ng/mL, about 1450 ng/mL or about 1500 ng/mL. In some aspects, the level of CD44 in the control sample is about 300 ng/mL.

In some aspects, the level of CD44 in the control sample is about 200 ng/mL to about 400 ng/mL. In some aspects, the level of CD44 in the control sample is about 200 ng/mL to about 350 ng/mL. In some aspects, the level of CD44 in the control sample is about 200 ng/mL to about 300 ng/mL. In some aspects, the level of CD44 in the control sample is about 200 ng/mL to about 250 ng/mL. In some aspects, the level of CD44 in the control sample is about 250 ng/mL to about 400 ng/mL. In some aspects, the level of CD44 in the control sample is about 300 ng/mL to about 400 ng/mL. In some aspects, the level of CD44 in the control sample is about 350 ng/mL to about 400 ng/mL.

In some aspects, the level of CD 147 in the control sample is about 50 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1050 ng/mL, about 1100 ng/mL, about 1150 ng/mL, about 1200 ng/mL, about 1250 ng/mL, about 1300 ng/mL, about 1350 ng/mL, about 1400 ng/mL, about 1450 ng/mL or about 1500 ng/mL. In some aspects, the level of CD147 in the control sample is about 700 ng/mL.

In some aspects, the level of CD147 in the control sample is about 600 ng/mL to about 750 ng/mL. In some aspects, the level of CD147 in the control sample is about 600 ng/mL to about 700 ng/mL. In some aspects, the level of CD147 in the control sample is about 600 ng/mL to about 650 ng/mL. In some aspects, the level of CD147 in the control sample is about 650 ng/mL to about 800 ng/mL. In some aspects, the level of CD147 in the control sample is about 700 ng/mL to about 800 ng/mL. In some aspects, the level of CD147 in the control sample is about 750 ng/mL to about 800 ng/mL.

In some aspects, the level of SPP1 in the control sample is about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1050 ng/mL, about 1100 ng/mL, about 1150 ng/mL, about 1200 ng/mL, about 1250 ng/mL, about 1300 ng/mL, about 1350 ng/mL, about 1400 ng/mL, about 1450 ng/mL or about 1500 ng/mL. In some aspects, the level of SPP1 in the control sample is about 50 ng/mL.

In some aspects. the level of SPP1 in the control sample is about 20 ng/mL to about 80 ng/mL. In some aspects, the level of SPP1 in the control sample is about 10 ng/mL to about 20 ng/mL, about 20 ng/mL to about 30 ng/mL, about 30 ng/mL to about 40 ng/mL, about 40 ng/mL to about 50 ng/mL, about 50 ng/mL to about 60 ng/mL, about 60 ng/mL to about 70 ng/mL, about 70 ng/mL to about 80 ng/mL, about 80 ng/mL to about 90 ng/mL, about 90 ng/mL to about 100 ng/mL, about 100 ng/mL to about 125 ng/mL, or about 125 ng/mL to about 150 ng/mL. In some aspects, the level of SPP1 in the control sample is about 20 ng/mL to about 80 ng/mL.

In some aspects, the method comprises administering a therapeutically effective amount of at least one additional therapeutic agent. In some aspects, the additional therapeutic agent is nintedanib or pirfenidone. In some aspects, the anti-Claudin-1 antibody or antigen binding fragment thereof and the at least one additional therapeutic agent is administered concurrently or sequentially.

### VIII. Kits

The present disclosure also provides kits, or products of manufacture, comprising an anti-Claudin-1 antibody and an ELISA kit. In some aspects, the ELISA kit measures soluble CD44. In some aspects, the ELISA kit measures soluble CD 147. In some aspects, the ELISA kit measures SPP1. In some aspects, the kit further comprises a lancet and a vial for obtaining a blood sample. In some aspects, the blood sample is a blood plasma sample.

One skilled in the art will readily recognize that the anti-Claudin-1 antibody of the present disclosure, can be readily incorporated into one of the established kit formats which are well known in the art.

The following examples are illustrative and do not limit the scope of the claimed aspects.

### EXAMPLES

### Example 1

### Soluble CD44 is a Biomarker for Anti-Claudin-1 Antibody Target Engagement

Anti-Claudin-1 monoclonal antibody binds to Claudin-1 (H1L1 antibody), which results in downstream activation of metalloproteinases that cleave CD44 into its soluble form (FIG. 1).

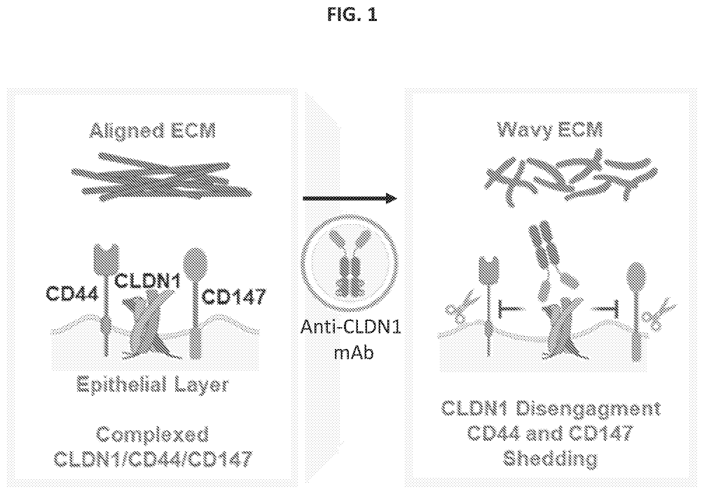

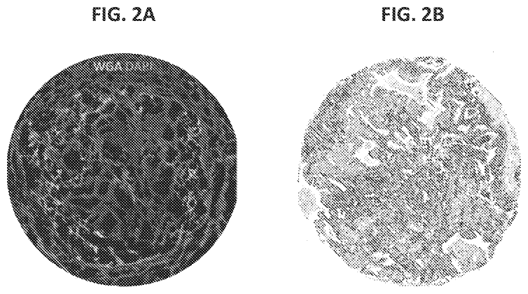

Human patients suffering from kidney fibrosis were enrolled in a Phase 1 study and treated with an anti-Claudin-1 antibody (H1L1 antibody) Kidney fibrosis samples were obtained from the patients. The kidney fibrosis samples were stained for both anti-Claudin-1 antibody and CD44. FIGS. 2A-2B shows co-localization of the anti-Claudin-1 antibody and CD44 via immunohistochemistry. There is also high CD44 expression in Lung Fibrosis. CD44 is also the receptor for Hyaluronic Acid, which is a component of ELF Score (used in Liver Fibrosis).

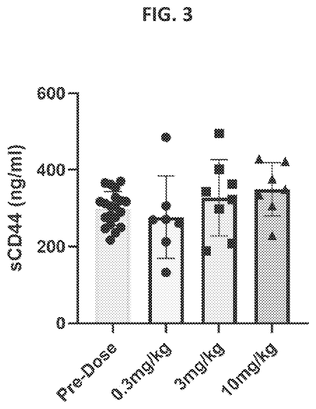

Next, patients were divided into single ascending dose cohorts (SAD) for anti-Claudin-1 antibody (H1L1 antibody) (pre-dose, 0.3 mg/kg, 3 mg/kg, and 10 mg/kg). Plasma samples were obtained from each cohort and soluble CD44 (ng/mL) was measured using ELISA (Abcam Human CD44 ELISA Kit (ab287168)) according to the manufacturer's manual. FIG. 3 shows that as the dose of anti-Claudin-1 antibody was increased, the level of soluble CD44 also increased.

### Example 2

### Soluble CD147 is a Biomarker for Anti-Claudin-1 Antibody Target Engagement

Anti-Claudin-1 monoclonal antibody binds to Claudin-1 (H1L1 antibody), which results in downstream activation of metalloproteinases that cleave CD147 into its soluble form (FIG. 1).

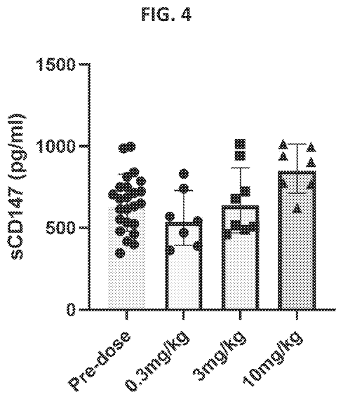

Human patients with kidney fibrosis were enrolled in a Phase 1 study and treated with an anti-Claudin-1 antibody (H1L1 antibody). Patients were divided into single ascending dose cohorts for anti-Claudin-1 antibody (H1L1 antibody) (pre-dose, 0.3 mg/kg, 3 mg/kg, and 10 mg/kg). Plasma samples were obtained from each cohort and soluble CD147 (ng/mL) was measured using ELISA (Abcam Human EMMPRIN ELISA Kit (ab219631)). FIG. 4 shows that as the dose of anti-Claudin-1 antibody was increased. the level of soluble CD147 also increased.

### Example 3

### Phase 1 Study-Decreased Plasma CD44 and SPP1 Following Treatment with Anti-Claudin-1 Monoclonal Antibody

Biomarkers measurements from Phase 1 healthy volunteers study have demonstrated decrease of plasmatic level of CD44 and SPP1 following treatment with anti-Claudin1 monoclonal antibody (H1L1 antibody).

### CD44 Biomarker

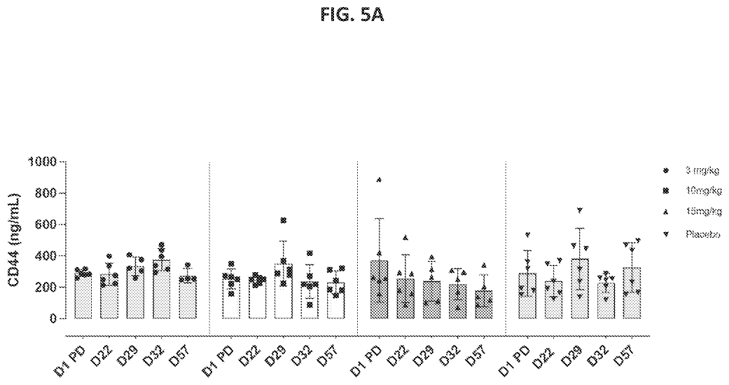

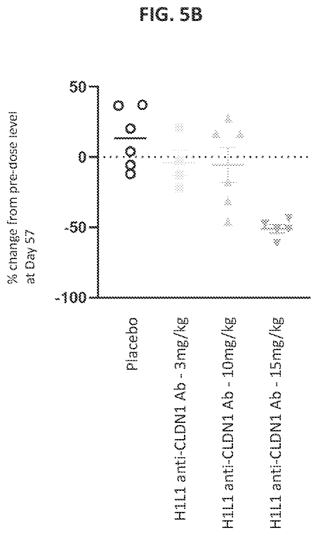

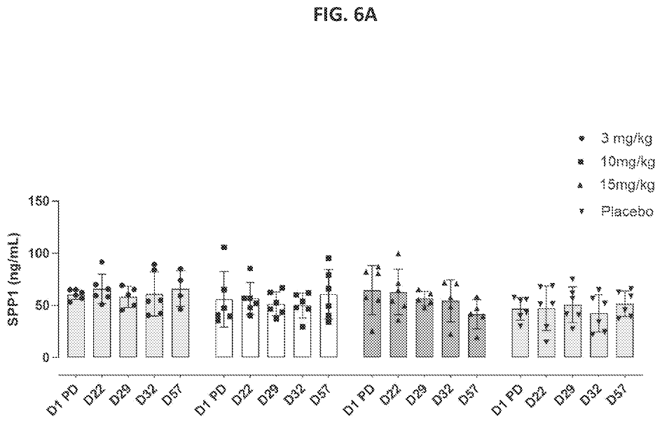

Patients were divided into multiple ascending dose cohorts (MAD) for treatment with anti-Claudin-1 antibody (H1L1 antibody) (FIGS. 5A and 5B) Plasma samples were obtained from each cohort (placebo-treated cohort and anti-Claudin-1 antibody treated cohort) and plasma CD44 (ng/mL) was measured using ELISA at various time points; Pre-Dose (D1 PD),Day 22 (D22), Day 29 (D29), Day 32 (D32) and Day 57 (D57). Patients have received 3 administrations of anti-Claudin-1 antibody or Placebo at Day 57.

At Day 57, plasma CD44 levels were not decreased in patients treated with 3 mg/kg of anti-Claudin-1 antibody, relative to the same patients at D1 PD (FIG. 5B), and relative to patients treated with placebo (FIG. 5A). At Day 57, plasma CD44 levels were not decreased in patients treated with 10 mg/kg of anti-Claudin-1 antibody, relative to the same patients at D1 PD (FIG. 5B), and relative to patients treated with placebo (FIG. 5A).

At D57, plasma CD44 levels were significantly decreased in patients treated with 15 mg/kg of anti-Claudin-1 antibody, relative to the same patients at D1 PD (FIG. 5B) (p-value <0.01), and relative to patients treated with placebo (FIG. 5A). Altogether, the data from the Phase 1 study demonstrates that treatment of humans with anti-Claudin-1 monoclonal antibody at certain doses results in a significant decrease in plasma CD44 levels.

Safety studies did not reveal any serious adverse events even at high steady-state concentrations. These results suggest the use of CLDN1-specific monoclonal antibodies for the treatment of fibrosis. Antifibrotic effects indicate a role of CLDN1 as a therapeutic target for tissue fibrosis across organs that CD44 plays a role in, such as liver, kidney and lung,

### SPP1

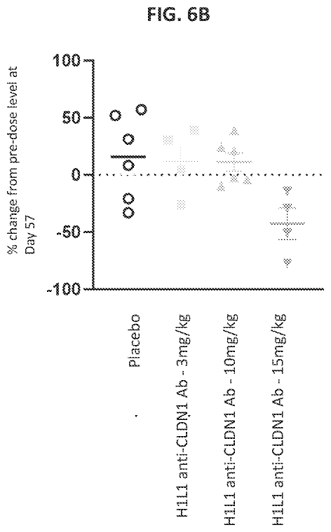

Patients were divided into multiple ascending dose cohorts (MAD) for treatment with anti-Claudin-1 antibody (H1L1 antibody) (FIGS. 6A and 6B). Plasma samples were obtained from each cohort (placebo-treated cohort and anti-Claudin-1 antibody treated cohort) and plasma SPP1 (ng/mL) was measured using ELISA (Human Osteopontin (OPN) Immunoassay R&D (Catalog Number DOST00)) at various time points; Pre-Dose (D1), Day 22 (D22), Day 29 (D29), Day 32 (D32) and Day 57 (D57). Patients have received 3 administrations of anti-Claudin-1 antibody or Placebo at Day 57.

At Day 57, plasma SPP1 levels were not decreased in patients treated with 3 mg/kg of anti-Claudin-1 antibody, relative to the same patients at D1 PD (FIG. 6B), and relative to patients treated with placebo (FIG. 6A). At Day 57, plasma SPP1 levels were not decreased in patients treated with 10 mg/kg of anti-Claudin-1 antibody, relative to the same patients at D1 PD (FIG. 6B), and relative to patients treated with placebo (FIG. 6A).

At D57, plasma SPP1 levels were significantly decreased in patients treated with 16 mg/kg of anti-Claudin-1 antibody, relative to the same patients at D1 PD (FIG. 6B) (p-value <0.01), and relative to patients treated with placebo (FIG. 6A). Altogether, the data from the Phase 1 study demonstrates that treatment of humans with anti-Claudin-1 monoclonal antibody at certain doses results in a significant decrease in plasma SPP1 levels.

Safety studies did not reveal any serious adverse events even at high steady-state concentrations. These results suggest the use of CLDN1-specific monoclonal antibodies for the treatment of fibrosis. Antifibrotic effects indicate a role of CLDN1 as a therapeutic target for tissue fibrosis across organs that SPP1 plays a role in, such as liver, kidney and lung.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

### SEQUENCES OF THE DISCLOSURE

TABLE 3
Sequences of the Disclosure

| SEQ ID NO: | Description | Amino Acid Sequence |
|---|---|---|
| SEQ ID NO: 1 | H1L1 - | EVQLVESGGGLVKPGGSLRLSCAASGFSFSSYGMNWVRQAPGKGL |
| | Heavy Chain | EWVSSISPSGSYFYYADSVKGRFTISRDNAKNSLYLQMNSLRAED |
| | #1 | TAVYYCARLPGFNPPFDHWGQGTLVTVSSASTKGPSVFPLAPSSK |
| | | STSGGTAALGCLVKDYFPEPVTVSWNSGALTSGVHTFPAVLQSSG |
| | | LYSLSSVVTVPSSSLGTQTYICNVNHKPSNTKVDKRVEPKSCDKT |
| | | HTCPPCPAPEFEGGPSVFLFPPKPKDTLMISRTPEVTCVVVDVSH |
| | | EDPEVKFNWYVDGVEVHNAKTKPREEQYNSTYRVVSVLTVLHQDW |
| | | LNGKEYKCKVSNKALPASIEKTISKAKGQPREPQVYTLPPSREEM |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| SEQ ID NO: 2 | H1L1 - Light | DIQMTQSPATLSVSPGERATLSCKASQNVGGNVDWYQWKPGQAPR |
| | Chain | LLIYGASNRYTGIPARFRGSGSGTEFTLTISSLQSEDFAVYYCLQ |
| | | YKNNPWTFGQGTKVEIKRTVAAPSVFIFPPSDEQLKSGTASVVCL |
| | | INNFYPREAKVQWKVDNALQSGNSQESVTEQDSKDSTYSLSSTLT |
| | | LSKADYEKHKVYACEVTHQGLSSPVTKSFNRGEC |
| SEQ ID NO: 3 | H1L1 - VH | EVOLVESGGGLVKPGGSLRLSCAASGFSFSSYGMNWVRQAPGKGL |
| | | EWVSSISPSGSYFYYADSVKGRFTISRDNAKNSLYLQMNSLRAED |
| | | TAVYYCARLPGFNPPFDHWGQGTLVTVSS |
| SEQ ID NO: 4 | H1L1 - VL | DIQMTQSPATLSVSPGERATLSCKASQNVGGNVDWYQWKPGQAPR |
| | | LLIYGASNRYTGIPARFRGSGSGTEFTLTISSLQSEDFAVYYCLQ |
| | | YKNNPWTFGQGTKVEIK |
| SEQ ID NO: 5 | HQL1/H3L3 - | GFSFSSYG |
| | CDR H1 | |
| SEQ ID NO: 6 | H1L1/H3L3 - | ISPSGSYF |
| | CDR H2 | |
| SEQ ID NO: 7 | H1L1/H3L3 - | ARLPGFNPPFDH |
| | CDR H3 | |
| SEQ ID NO: 8 | H1L1/H3L3 - | QNVGGN |
| | CDR L1 | |
| n/a | H1L1/H3L3 - | GA |
| | CDR L2 | |
| SEQ ID NO: 10 | H1L1/H3L3 - | LQYKNNPWT |
| | CDR L3 | |
| SEQ ID NO: 11 | H3L3 - | QVQLVESGGGVVQPGRSLRLSCLGSGFSFSSYGMNWVRQAPGKGL |
| | Heavy Chain | EWVASISPSGSYFYYADSVKGRFTISRDNSKNTLYLQMTSLRAED |
| | | TAIYYCARLPGFNPPFDHWGQGTLVTVSSASTKGPSVFPLAPSSK |
| | | STSGGTAALGCLVKDYFPEPVTVSWNSGALTSGVHTFPAVLQSSG |
| | | LYSLSSVVTVPSSSLGTQTYICNVNHKPSNTKVDKRVEPKSCDKT |
| | | HTCPPCPAPELLGGPSVFLFPPKPKDTLMISRTPEVTCVVVDVSH |
| | | EDPEVKFNWYVDGVEVHNAKTKPREEQYNSTYRVVSVLTVLHQDW |
| | | LNGKEYKCKVSNKALPAPIEKTISKAKGQPREPQVYTLPPSREEM |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| SEQ ID NO: 12 | H3L3- Light | DIQMTQSPSSLSASVGDRVTITCKASQNVGGNVDWYQWKPGKAPK |
| | Chain | LLIYGASNRYTGVPDRFRGSGSGTDFTLTISSLQPEDVATYYCLQ |
| | | YKNNPWTFGGGTKVEIKRTVAAPSVFIFPPSDEQLKSGTASVVCL |
| | | LNNFYPREAKVQWKVDNALQSGNSQESVTEQDSKDSTYSLSSTLT |
| | | LSKADYEKHKVYACEVTHQGLSSPVTKSFNRGEC |
| SEQ ID NO: 13 | H3L3 - VH | QVQLVESGGGVVQPGRSLRLSCLGSGFSFSSYGMNWVRQAPGKGL |
| | | EWVASISPSGSYFYYADSVKGRFTISRDNSKNTLYLQMTSLRAED |
| | | TAIYYCARLPGFNPPFDHWGQGTLVTVSS |
| SEQ ID NO: 14 | H3L3 - VL | DIQMTQSPSSLSASVGDRVTITCKASQNVGGNVDWYQWKPGKAPK |
| | | LLIYGASNRYTGVPDRFRGSGSGTDFTLTISSLOPEDVATYYCLQ |
| | | YKNNPWTFGGGTKVEIK |
| SEQ ID NO: 15 | human CD44 | CTCATTGCCCAGCGGACCCCAGCCTCTGCCAGGTTCGGTCCGCCA |
| | mRNA | TCCTCGTCCCGTCCTCCGCCGGCCCCTGCCCCGCGCCCAGGGATC |
| | sequence | CTCCAGCTCCTTTCGCCCGCGCCCTCCGTTCGCTCCGGACACCAT |
| | | GGACAAGTTTTGGTGGCACGCAGCCTGGGGACTCTGCCTCGTGCC |
| | | GCTGAGCCTGGCGCAGATCGATTTGAATATAACCTGCCGCTTTGC |
| | | AGGTGTATTCCACGTGGAGAAAAATGGTCGCTACAGCATCTCTCG |
| | | GACGGAGGCCGCTGACCTCTGCAAGGCTTTCAATAGCACCTTGCC |
| | | CACAATGGCCCAGATGGAGAAAGCTCTGAGCATCGGATTTGAGAC |
| | | CTGCAGGTATGGGTTCATAGAAGGGCACGTGGTGATTCCCCGGAT |
| | | CCACCCCAACTCCATCTGTGCAGCAAACAACACAGGGGTGTACAT |
| | | CCTCACATCCAACACCTCCCAGTATGACACATATTGCTTCAATGC |
| | | TTCAGCTCCACCTGAAGAAGATTGTACATCAGTCACAGACCTGCC |
| | | CAATGCCTTTGATGGACCAATTACCATAACTATTGTTAACCGTGA |
| | | TGGCACCCGCTATGTCCAGAAAGGAGAATACAGAACGAATCCTGA |
| | | AGACATCTACCCCAGCAACCCTACTGATGATGACGTGAGCAGCGG |
| | | CTCCTCCAGTGAAAGGAGCAGCACTTCAGGAGGTTACATCTTTTA |
| | | CACCTTTTCTACTGTACACCCCATCCCAGACGAAGACAGTCCCTG |
| | | GATCACCGACAGCACAGACAGAATCCCTGCTACCACTTTGATGAG |
| | | CACTAGTGCTACAGCAACTGAGACAGCAACCAAGAGGCAAGAAAC |
| | | CTGGGATTGGTTTTCATGGTTGTTTCTACCATCAGAGTCAAAGAA |
| | | TCATCTTCACACAACAACACAAATGGCTGGTACGTCTTCAAATAC |
| | | CATCTCAGCAGGCTGGGAGCCAAATGAAGAAAATGAAGATGAAAG |
| | | AGACAGACACCTCAGTTTTTCTGGATCAGGCATTGATGATGATGA |
| | | AGATTTTATCTCCAGCACCATTTCAACCACACCACGGGCTTTTGA |
| | | CCACACAAAACAGAACCAGGACTGGACCCAGTGGAACCCAAGCCA |
| | | TTCAAATCCGGAAGTGCTACTTCAGACAACCACAAGGATGACTGA |
| | | TGTAGACAGAAATGGCACCACTGCTTATGAAGGAAACTGGAACCC |
| | | AGAAGCACACCCTCCCCTCATTCACCATGAGCATCATGAGGAAGA |
| | | AGAGACCCCACATTCTACAAGCACAATCCAGGCAACTCCTAGTAG |
| | | TACAACGGAAGAAACAGCTACCCAGAAGGAACAGTGGTTTGGCAA |
| | | CAGATGGCATGAGGGATATCGCCAAACACCCAAAGAAGACTCCCA |
| | | TTCGACAACAGGGACAGCTGCAGCCTCAGCTCATACCAGCCATCC |
| | | AATGCAAGGAAGGACAACACCAAGCCCAGAGGACAGTTCCTGGAC |
| | | TGATTTCTTCAACCCAATCTCACACCCCATGGGACGAGGTCATCA |
| | | AGCAGGAAGAAGGATGGATATGGACTCCAGTCATAGTATAACGCT |
| | | TCAGCCTACTGCAAATCCAAACACAGGTTTGGTGGAAGATTTGGA |
| | | CAGGACAGGACCTCTTTCAATGACAACGCAGCAGAGTAATTCTCA |
| | | GAGCTTCTCTACATCACATGAAGGCTTGGAAGAAGATAAAGACCA |
| | | TCCAACAACTTCTACTCTGACATCAAGCAATAGGAATGATGTCAC |
| | | AGGTGGAAGAAGAGACCCAAATCATTCTGAAGGCTCAACTACTTT |
| | | ACTGGAAGGTTATACCTCTCATTACCCACACACGAAGGAAAGCAG |
| | | GACCTTCATCCCAGTGACCTCAGCTAAGACTGGGTCCTTTGGAGT |
| | | TACTGCAGTTACTGTTGGAGATTCCAACTCTAATGTCAATCGTTC |
| | | CTTATCAGGAGACCAAGACACATTCCACCCCAGTGGGGGGTCCCA |
| | | TACCACTCATGGATCTGAATCAGATGGACACTCACATGGGAGTCA |
| | | AGAAGGTGGAGCAAACACAACCTCTGGTCCTATAAGGACACCCCA |
| | | AATTCCAGAATGGCTGATCATCTTGGCATCCCTCTTGGCCTTGGC |
| | | TTTGATTCTTGCAGTTTGCATTGCAGTCAACAGTCGAAGAAGGTG |
| | | TGGGCAGAAGAAAAAGCTAGTGATCAACAGTGGCAATGGAGCTGT |
| | | GGAGGACAGAAAGCCAAGTGGACTCAACGGAGAGGCCAGCAAGTC |
| | | TCAGGAAATGGTGCATTTGGTGAACAAGGAGTCGTCAGAAACTCC |
| | | AGACCAGTTTATGACAGCTGATGAGACAAGGAACCTGCAGAATGT |
| | | GGACATGAAGATTGGGGTGTAACACCTACACCATTATCTTGGAAA |
| | | GAAACAACCGTTGGAAACATAACCATTACAGGGAGCTGGGACACT |
| | | TAACAGATGCAATGTGCTACTGATTGTTTCATTGCGAATCTTTTT |
| | | TAGCATAAAATTTTCTACTCTTTTTGTTTTTTGTGTTTTGTTCTT |
| | | TAAAGTCAGGTCCAATTTGTAAAAACAGCATTGCTTTCTGAAATT |
| | | AGGGCCCAATTAATAATCAGCAAGAATTTGATCGTTCCAGTTCCC |
| | | ACTTGGAGGCCTTTCATCCCTCGGGTGTGCTATGGATGGCTTCTA |
| | | ACAAAAACTACACATATGTATTCCTGATCGCCAACCTTTCCCCCA |
| | | CCAGCTAAGGACATTTCCCAGGGTTAATAGGGCCTGGTCCCTGGG |
| | | AGGAAATTTGAATGGGTCCATTTTGCCCTTCCATAGCCTAATCCC |
| | | TGGGCATTGCTTTCCACTGAGGTTGGGGGTTGGGGTGTACTAGTT |
| | | ACACATCTTCAACAGACCCCCTCTAGAAATTTTTCAGATGCTTCT |
| | | GGGAGACACCCAAAGGGTGAAGCTATTTATCTGTAGTAAACTATT |
| | | TATCTGTGTTTTTGAAATATTAAACCCTGGATCAGTCCTTTGATC |
| | | AGTATAATTTTTTAAAGTTACTTTGTCAGAGGCACAAAAGGGTTT |
| | | AAACTGATTCATAATAAATATCTGTACTTCTTCGATCTTCACCTT |
| | | TTGTGCTGTGATTCTTCAGTTTCTAAACCAGCACTGTCTGGGTCC |
| | | CTACAATGTATCAGGAAGAGCTGAGAATGGTAAGGAGACTCTTCT |
| | | AAGTCTTCATCTCAGAGACCCTGAGTTCCCACTCAGACCCACTCA |
| | | GCCAAATCTCATGGAAGACCAAGGAGGGCAGCACTGTTTTTGTTT |
| | | TTTGTTTTTTGTTTTTTTTTTTTGACACTGTCCAAAGGTTTTCCA |
| | | TCCTGTCCTGGAATCAGAGTTGGAAGCTGAGGAGCTTCAGCCTCT |
| | | TTTATGGTTTAATGGCCACCTGTTCTCTCCTGTGAAAGGCTTTGC |
| | | AAAGTCACATTAAGTTTGCATGACCTGTTATCCCTGGGGCCCTAT |
| | | TTCATAGAGGCTGGCCCTATTAGTGATTTCCAAAAACAATATGGA |
| | | AGTGCCTTTTGATGTCTTACAATAAGAGAAGAAGCCAATGGAAAT |
| | | GAAAGAGATTGGCAAAGGGGAAGGATGATGCCATGTAGATCCTGT |
| | | TTGACATTTTTATGGCTGTATTTGTAAACTTAAACACACCAGTGT |
| | | CTGTTCTTGATGCAGTTGCTATTTAGGATGAGTTAAGTGCCTGGG |
| | | GAGTCCCTCAAAAGGTTAAAGGGATTCCCATCATTGGAATCTTAT |
| | | CACCAGATAGGCAAGTTTATGACCAAACAAGAGAGTACTGGCTTT |
| | | ATCCTCTAACCTCATATTTTCTCCCACTTGGCAAGTCCTTTGTGG |
| | | CATTTATTCATCAGTCAGGGTGTCCGATTGGTCCTAGAACTTCCA |
| | | AAGGCTGCTTGTCATAGAAGCCATTGCATCTATAAAGCAACGGCT |
| | | CCTGTTAAATGGTATCTCCTTTCTGAGGCTCCTACTAAAAGTCAT |
| | | TTGTTACCTAAACTTATGTGCTTAACAGGCAATGCTTCTCAGACC |
| | | ACAAAGCAGAAAGAAGAAGAAAAGCTCCTGACTAAATCAGGGCTG |
| | | GGCTTAGACAGAGTTGATCTGTAGAATATCTTTAAAGGAGAGATG |
| | | TCAACTTTCTGCACTATTCCCAGCCTCTGCTCCTCCCTGTCTACC |
| | | CTCTCCCCTCCCTCTCTCCCTCCACTTCACCCCACAATCTTGAAA |
| | | AACTTCCTTTCTCTTCTGTGAACATCATTGGCCAGATCCATTTTC |
| | | AGTGGTCTGGATTTCTTTTTATTTTCTTTTCAACTTGAAAGAAAC |
| | | TGGACATTAGGCCACTATGTGTTGTTACTGCCACTAGTGTTCAAG |
| | | TGCCTCTTGTTTTCCCAGAGATTTCCTGGGTCTGCCAGAGGCCCA |
| | | GACAGGCTCACTCAAGCTCTTTAACTGAAAAGCAACAAGCCACTC |
| | | CAGGACAAGGTTCAAAATGGTTACAACAGCCTCTACCTGTCGCCC |
| | | CAGGGAGAAAGGGGTAGTGATACAAGTCTCATAGCCAGAGATGGT |
| | | TTTCCACTCCTTCTAGATATTCCCAAAAAGAGGCTGAGACAGGAG |
| | | GTTATTTTCAATTTTATTTTGGAATTAAATACTTTTTTCCCTTTA |
| | | TTACTGTTGTAGTCCCTCACTTGGATATACCTCTGTTTTCACGAT |
| | | AGAAATAAGGGAGGTCTAGAGCTTCTATTCCTTGGCCATTGTCAA |
| | | CGGAGAGCTGGCCAAGTCTTCACAAACCCTTGCAACATTGCCTGA |
| | | AGTTTATGGAATAAGATGTATTCTCACTCCCTTGATCTCAAGGGC |
| | | GTAACTCTGGAAGCACAGCTTGACTACACGTCATTTTTACCAATG |
| | | ATTTTCAGGTGACCTGGGCTAAGTCATTTAAACTGGGTCTTTATA |
| | | AAAGTAAAAGGCCAACATTTAATTATTTTGCAAAGCAACCTAAGA |
| | | GCTAAAGATGTAATTTTTCTTGCAATTGTAAATCTTTTGTGTCTC |
| | | CTGAAGACTTCCCTTAAAATTAGCTCTGAGTGAAAAATCAAAAGA |
| | | GACAAAAGACATCTTCGAATCCATATTTCAAGCCTGGTAGAATTG |
| | | GCTTTTCTAGCAGAACCTTTCCAAAAGTTTTATATTGAGATTCAT |
| | | AACAACACCAAGAATTGATTTTGTAGCCAACATTCATTCAATACT |
| | | GTTATATCAGAGGAGTAGGAGAGAGGAAACATTTGACTTATCTGG |
| | | AAAAGCAAAATGTACTTAAGAATAAGAATAACATGGTCCATTCAC |
| | | CTTTATGTTATAGATATGTCTTTGTGTAAATCATTTGTTTTGAGT |
| | | TTTCAAAGAATAGCCCATTGTTCATTCTTGTGCTGTACAATGACC |
| | | ACTGTTATTGTTACTTTGACTTTTCAGAGCACACCCTTCCTCTGG |
| | | TTTTTGTATATTTATTGATGGATCAATAATAATGAGGAAAGCATG |
| | | ATATGTATATTGCTGAGTTGAAAGCACTTATTGGAAAATATTAAA |
| | | AGGCTAACATTAAAAGACTAAAGGAAACAGA |
| SEQ ID NO: 16 | human CD44 | MDKFWWHAAWGLCLVPLSLAQIDLNITCRFAGVFHVEKNGRYSIS |
| | AA | RTEAADLCKAFNSTLPTMAQMEKALSIGFETCRYGFIEGHVVIPR |
| | | IHPNSICAANNTGVYILTSNTSQYDTYCFNASAPPEEDCTSVTDL |
| | | PNAFDGPITITIVNRDGTRYVQKGEYRINPEDIYPSNPTDDDVSS |
| | | GSSSERSSTSGGYIFYTFSTVHPIPDEDSPWITDSTDRIPATTLM |
| | | STSATATETATKRQETWDWFSWLFLPSESKNHLHTTTQMAGTSSN |
| | | TISAGWEPNEENEDERDRHLSFSGSGIDDDEDFISSTISTTPRAF |
| | | DHTKQNQDWTQWNPSHSNPEVLLQTTTRMTDVDRNGTTAYEGNWN |
| | | PEAHPPLIHHEHHEEEETPHSTSTIQATPSSTTEETATQKEQWFG |
| | | NRWHEGYRQTPKEDSHSTTGTAAASAHTSHPMQGRTTPSPEDSSW |
| | | TDFFNPISHPMGRGHQAGRRMDMDSSHSITLQPTANPNTGLVEDL |
| | | DRTGPLSMTTQQSNSQSFSTSHEGLEEDKDHPTTSTLTSSNRNDV |
| | | TGGRRDPNHSEGSTTLLEGYTSHYPHTKESRTFIPVTSAKTGSFG |
| | | VTAVTVGDSNSNVNRSLSGDQDTFHPSGGSHTTHGSESDGHSHGS |
| | | QEGGANTTSGPIRTPQIPEWLIILASLLALALILAVCIAVNSRRR |
| | | CGQKKKLVINSGNGAVEDRKPSGLNGEASKSQEMVHLVNKESSET |
| | | PDQFMTADETRNLQNVDMKIGV |
| SEQ ID NO: 17 | human | AGCGGTTGGAGGTTGTAGGACCGGCGAGGAATAGGAATCATGGCG |
| | CD147 | GCTGCGCTGTTCGTGCTGCTGGGATTCGCGCTGCTGGGCACCCAC |
| | mRNA | GGAGCCTCCGGGGCTGCCGGCACAGTCTTCACTACCGTAGAAGAC |
| | | CTTGGCTCCAAGATACTCCTCACCTGCTCCTTGAATGACAGCGCC |
| | | ACAGAGGTCACAGGGCACCGCTGGCTGAAGGGGGGCGTGGTGCTG |
| | | AAGGAGGACGCGCTGCCCGGCCAGAAAACGGAGTTCAAGGTGGAC |
| | | TCCGACGACCAGTGGGGAGAGTACTCCTGCGTCTTCCTCCCCGAG |
| | | CCCATGGGCACGGCCAACATCCAGCTCCACGGGCCTCCCAGAGTG |
| | | AAGGCTGTGAAGTCGTCAGAACACATCAACGAGGGGGAGACGGCC |
| | | ATGCTGGTCTGCAAGTCAGAGTCCGTGCCACCTGTCACTGACTGG |
| | | GCCTGGTACAAGATCACTGACTCTGAGGACAAGGCCCTCATGAAC |
| | | GGCTCCGAGAGCAGGTTCTTCGTGAGTTCCTCGCAGGGCCGGTCA |
| | | GAGCTACACATTGAGAACCTGAACATGGAGGCCGACCCCGGCCAG |
| | | TACCGGTGCAACGGCACCAGCTCCAAGGGCTCCGACCAGGCCATC |
| | | ATCACGCTCCGCGTGCGCAGCCACCTGGCCGCCCTCTGGCCCTTC |
| | | CTGGGCATCGTGGCTGAGGTGCTGGTGCTGGTCACCATCATCTTC |
| | | ATCTACGAGAAGCGCCGGAAGCCCGAGGACGTCCTGGATGATGAC |
| | | GACGCCGGCTCTGCACCCCTGAAGAGCAGCGGGCAGCACCAGAAT |
| | | GACAAAGGCAAGAACGTCCGCCAGAGGAACTCTTCCTGAGGTGGC |
| | | CCGAGGACGCTCCCTGCTCCACGTCTGCGCCGCCGCCGGAGTCCA |
| | | CTCCCAGTGCTTGCAAGATTCCAAGTTCTCACCTCTTAAAGAAAA |
| | | CCCACCCCGTAGATTCCCATCATACACTTCCTTCTTTTTTAAAAA |
| | | AGTTGGGTTTTCTCCATTCAGGATTCTGTTCCTTAGGTTTTTTTC |
| | | CTTCTGAAGTGTTTCACGAGAGCCCGGGAGCTGCTGCCCTGCGGC |
| | | CCCGTCTGTGGCTTTCAGCCTCTGGGTCTGAGTCATGGCCGGGTG |
| | | GGCGGCACAGCCTTCTCCACTGGCCGGAGTCAGTGCCAGGTCCTT |
| | | GCCCTTTGTGGAAAGTCACAGGTCACACGAGGGGCCCCGTGTCCT |
| | | GCCTGTCTGAAGCCAATGCTGTCTGGTTGCGCCATTTTTGTGCTT |
| | | TTATGTTTAATTTTATGAGGGCCACGGGTCTGTGTTCGACTCAGC |
| | | CTCAGGGACGACTCTGACCTCTTGGCCACAGAGGACTCACTTGCC |
| | | CACACCGAGGGCGACCCCGTCACAGCCTCAAGTCACTCCCAAGCC |
| | | CCCTCCTTGTCTGTGCATCCGGGGGCAGCTCTGGAGGGGGTTTGC |
| | | TGGGGAACTGGCGCCATCGCCGGGACTCCAGAACCGCAGAAGCCT |
| | | CCCCAGCTCACCCCTGGAGGACGGCCGGCTCTCTATAGCACCAGG |
| | | GCTCACGTGGGAACCCCCCTCCCACCCACCGCCACAATAAAGATC |
| | | GCCCCCACCTCCACCCTCA |
| SEQ ID NO: 18 | human | MAAALFVLLGFALLGTHGASGAAGTVFTTVEDLGSKILLTCSLND |
| | CD147 AA | SATEVTGHRWLKGGVVLKEDALPGQKTEFKVDSDDQWGEYSCVFL |
| | | PEPMGTANIQLHGPPRVKAVKSSEHINEGETAMLVCKSESVPPVT |
| | | DWAWYKITDSEDKALMNGSESRFFVSSSQGRSELHIENLNMEADP |
| | | GQYRCNGTSSKGSDQAIITLRVRSHLAALWPFLGIVAEVLVLVTI |
| | | IFIYEKRRKPEDVLDDDDAGSAPLKSSGQHQNDKGKNVRQRNSS |
| SEQ ID NO: 26 | Human SPP1 | AGCAGCAGGAGGAGGCAGAGCACAGCATCGTCGGGACCAGACTCG |
| | mRNA | TCTCAGGCCAGTTGCAGCCTTCTCAGCCAAACGCCGACCAAGGAA |
| | | AACTCACTACCATGAGAATTGCAGTGATTTGCTTTTGCCTCCTAG |
| | | GCATCACCTGTGCCATACCAGTTAAACAGGCTGATTCTGGAAGTT |
| | | CTGAGGAAAAGCAGCTTTACAACAAATACCCAGATGCTGTGGCCA |
| | | CATGGCTAAACCCTGACCCATCTCAGAAGCAGAATCTCCTAGCCC |
| | | CACAGACCCTTCCAAGTAAGTCCAACGAAAGCCATGACCACATGG |
| | | ATGATATGGATGATGAAGATGATGATGACCATGTGGACAGCCAGG |
| | | ACTCCATTGACTCGAACGACTCTGATGATGTAGATGACACTGATG |
| | | ATTCTCACCAGTCTGATGAGTCTCACCATTCTGATGAATCTGATG |
| | | AACTGGTCACTGATTTTCCCACGGACCTGCCAGCAACCGAAGTTT |
| | | TCACTCCAGTTGTCCCCACAGTAGACACATATGATGGCCGAGGTG |
| | | ATAGTGTGGTTTATGGACTGAGGTCAAAATCTAAGAAGTTTCGCA |
| | | GACCTGACATCCAGTACCCTGATGCTACAGACGAGGACATCACCT |
| | | CACACATGGAAAGCGAGGAGTTGAATGGTGCATACAAGGCCATCC |
| | | CCGTTGCCCAGGACCTGAACGCGCCTTCTGATTGGGACAGCCGTG |
| | | GGAAGGACAGTTATGAAACGAGTCAGCTGGATGACCAGAGTGCTG |
| | | AAACCCACAGCCACAAGCAGTCCAGATTATATAAGCGGAAAGCCA |
| | | ATGATGAGAGCAATGAGCATTCCGATGTGATTGATAGTCAGGAAC |
| | | TTTCCAAAGTCAGCCGTGAATTCCACAGCCATGAATTTCACAGCC |
| | | ATGAAGATATGCTGGTTGTAGACCCCAAAAGTAAGGAAGAAGATA |
| | | AACACCTGAAATTTCGTATTTCTCATGAATTAGATAGTGCATCTT |
| | | CTGAGGTCAATTAAAAGGAGAAAAAATACAATTTCTCACTTTGCA |
| | | TTTAGTCAAAAGAAAAAATGCTTTATAGCAAAATGAAAGAGAACA |
| | | TGAAATGCTTCTTTCTCAGTTTATTGGTTGAATGTGTATCTATTT |
| | | GAGTCTGGAAATAACTAATGTGTTTGATAATTAGTTTAGTTTGTG |
| | | GCTTCATGGAAACTCCCTGTAAACTAAAAGCTTCAGGGTTATGTC |
| | | TATGTTCATTCTATAGAAGAAATGCAAACTATCACTGTATTTTAA |
| | | TATTTGTTATTCTCTCATGAATAGAAATTTATGTAGAAGCAAACA |
| | | AAATACTTTTACCCACTTAAAAAGAGAATATAACATTTTATGTCA |
| | | CTATAATCTTTTGTTTTTTAAGTTAGTGTATATTTTGTTGTGATT |
| | | ATCTTTTTGTGGTGTGAATAAATCTTTTATCTTGAATGTAATAAG |
| | | AATTTGGTGGTGTCAATTGCTTATTTGTTTTCCCACGGTTGTCCA |
| | | GCAATTAATAAAACATAACCTTTTTTACTGCCTA |
| SEQ ID NO: 27 | Human SPP1 | MRIAVICFCLLGITCAIPVKQADSGSSEEKQLYNKYPDAVATWLN |
| | AA | PDPSQKQNLLAPQTLPSKSNESHDHMDDMDDEDDDDHVDSQDSID |
| | | SNDSDDVDDTDDSHQSDESHHSDESDELVTDFPTDLPATEVFTPV |
| | | VPTVDTYDGRGDSVVYGLRSKSKKFRRPDIQYPDATDEDITSHME |
| | | SEELNGAYKAIPVAQDLNAPSDWDSRGKDSYETSQLDDQSAETHS |
| | | HKQSRLYKRKANDESNEHSDVIDSQELSKVSREFHSHEFHSHEDM |
| | | LVVDPKSKEEDKHLKFRISHELDSASSEVN |
| SEQ ID NO: 19 | CD3zeta | RVKFSRSADAPAYKQGQNQLYNELNLGRREEYDVLDKRRGRDPEM |
| | signaling | GGKPRRKNPQEGLYNELQKDKMAEAYSEIGMKGERRRGKGHDGLY |
| | domain | QGLSTATKDTYDALHMQALPPR |
| SEQ ID NO: 20 | (G4S) linker | GGGGSGGGGSGGGGS |
| SEQ ID NO: 21 | linker 2 | GSTSGSGKPGSGEGSTKG |
| SEQ ID NO: 22 | CD8alpha | TTTPAPRPPTPAPTIASQPLSLRPEACRPAAGGAVHTRGLDFACD |
| | hinge | |
| SEQ ID NO: 23 | TM domain | IYIWAPLAGTCGVLLLSLVITLYC |
| SEQ ID NO: 24 | costimulatory | KRGRKKLLYIFKQPFMRPVQTTQEEDGCSCRFPEEEEGGCEL |
| | domain | |
| SEQ ID NO: 25 | H1L1 Heavy | EVQLVESGGGLVKPGGSLRLSCAASGFSFSSYGMNWVRQAPGKGL |
| | Chain #2 | EWVSSISPSGSYFYYADSVKGRFTISRDNAKNSLYLQMNSLRAED |
| | | TAVYYCARLPGFNPPFDHWGQGTLVTVSSASTKGPSVFPLAPSSK |
| | | STSGGTAALGCLVKDYFPEPVTVSWNSGALTSGVHTFPAVLQSSG |
| | | LYSLSSVVTVPSSSLGTQTYICNVNHKPSNTKVDKRVEPKSCDKT |
| | | HTCPPCPAPELLGGPSVFLFPPKPKDTLMISRTPEVTCVVVDVSH |
| | | EDPEVKFNWYVDGVEVHNAKTKPREEQYNSTYRVVSVLTVLHQDW |
| | | LNGKEYKCKVSNKALPAPIEKTISKAKGQPREPQVYTLPPSREEM |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSP |

### Embodiments

Embodiment 1. A method of treating fibrosis comprising:
(a) administering an anti-Claudin-1 antibody to a subject;
(b) determining a level of soluble CD44 in a test sample from the subject; and
(c) comparing the level of soluble CD44 in the test sample to a level of soluble CD44 in a control sample;
wherein if the level of soluble CD44 in the test sample is not increased relative to level of soluble CD44 in the control sample, then the dose of the anti-Claudin-1 antibody being administered to the subject is increased; wherein if the level of soluble CD44 in the test sample is increased relative to the control sample, then the dose of the anti-Claudin-1 antibody being administered to the subject is not increased.

Embodiment 2. A method of treating fibrosis in a subject, wherein the subject bas been determined to have a level of soluble CD44 in a test sample from the subject that is not increased relative to a level of soluble CD44 in a control sample, wherein the subject has previously been administered an anti-Claudin-1 treatment, the method comprising administering an increased dose of the anti-Claudin-1 treatment to the subject.

Embodiment 3. A method of monitoring fibrosis progression in a subject comprising: (a) quantifying a level of soluble CD44 in a test sample from the subject, wherein the subject has previously been administered an anti-Claudin-1 treatment; and (b) comparing the level of soluble CD44 in the test sample to a level of soluble CD44 in a control sample; wherein a level of soluble CD44 in the test sample that is not increased relative to the level of soluble CD44 in the control sample indicates that the subject is susceptible to an increased dose of the anti-Claudin-1 treatment.

Embodiment 4. A method of monitoring fibrosis progression in a subject comprising:
(a) determining a level of soluble CD44 in a test sample from the subject, wherein the subject has previously been administered an anti-Claudin-1 antibody; and
(b) comparing the level of soluble CD44 in the test sample to a level of soluble CD44 in a control sample; wherein a level of soluble CD44 in the test sample that is not increased relative to the level of soluble CD44 in the control sample indicates that the subject is susceptible to an increased dose of the anti-Claudin-1 antibody.

Embodiment 5. A method for treating a subject suffering from fibrosis, the method comprising the steps of: determining whether the subject has increased levels of soluble CD44 by obtaining a test sample from the subject and comparing the level of soluble CD44 in the test sample to a control sample, wherein the subject has previously been administered a dose of an anti-Claudin-1 antibody,
wherein if the subject has increased levels of soluble CD44 in the test sample relative to the control sample, then the subject is not administered an increased dose of the anti-Claudin-1 antibody;
wherein if the subject does not have increased levels of soluble CD44 in the test sample relative to the control sample, then the subject is administered an increased dose of the an anti-Claudin-1 antibody.

Embodiment 6. A method for designing a customized therapy for a subject suffering from fibrosis which comprises:
(a) quantifying the level of soluble CD44 in a test sample from the subject. wherein the subject has previously been administered a dose of an anti-Claudin-1 antibody;
(b) comparing the level of soluble CD44 in the test sample obtained in step (a) with the level of soluble CD44 in a control sample, wherein if the level of soluble CD44 in the test sample obtained in step (a) is not increased relative to the level of soluble CD44 in the control sample, then the subject is susceptible to receive an increased dose of the anti-Claudin-1 antibody;
(c) determining that the subject has a not increased level of soluble CD44 with respect to the level of soluble CD44 in the control sample; and
(d) administering an increased dose of the anti-Claudin-1 antibody.

Embodiment 7. A method of classifying a subject suffering from fibrosis into a cohort and treating said subject, comprising
(a) quantifying the level of soluble CD44 in a test sample from the subject, wherein the subject has previously been administered a dose of an anti-Claudin-1 antibody;
(b) comparing the level of soluble CD44 in the test sample obtained in step (a) with the level of soluble CD44 in a control sample, wherein if the level of soluble CD44 in the test sample obtained in step (a) is not increased relative to the level of soluble CD44 in the control sample, then the subject is susceptible to receive an increased dose of the anti-Claudin-1 antibody;
(c) determining that the subject has a not increased level of soluble CD44 with respect to the level of soluble CD44 in the control sample;
(d) classifying said subject into a cohort based on the level of soluble CD44 in the test sample relative to the control sample; and
(e) administering an increased dose of the anti-Claudin-1 antibody.

Embodiment 8. A method of treating fibrosis comprising:
(a) administering an anti-Claudin-1 antibody to a subject;
(b) determining a level of soluble CD147 in a test sample from the subject; and
(c) comparing the level of soluble CD147 in the test sample to a level of soluble CD147 in a control sample; wherein if the level of soluble CD147 in the test sample is not increased relative to level of soluble CD147 in the control sample, then the dose of the anti-Claudin-1 antibody being administered to the subject is increased; wherein if the level of soluble CD147 in the test sample is increased relative to the control sample, then the dose of the anti-Claudin-1 antibody being administered to the subject is not increased.

Embodiment 9. A method of treating fibrosis in a subject, wherein the subject has been determined to have a level of soluble CD147 in a test sample from the subject that is not increased relative to a level of soluble CD147 in a control sample, wherein the subject has previously been administered an anti-Claudin-1 treatment, the method comprising administering an increased dose of the anti-Claudin-1 treatment to the subject.

Embodiment 10. A method of monitoring fibrosis progression in a subject comprising:
(a) quantifying a level of soluble CD147 in a test sample from the subject, wherein the subject has previously been administered an anti-Claudin-1 treatment; and
(b) comparing the level of soluble CD147 in the test sample to a level of soluble CD147 in a control sample;
wherein a level of soluble CD147 in the test sample that is not increased relative to the level of soluble CD147 in the control sample indicates that the subject is susceptible to an increased dose of the anti-Claudin-1 treatment.

Embodiment 11. A method of monitoring fibrosis progression in a subject comprising:
(a) determining a level of soluble CD147 in a test sample from the subject, wherein the subject has previously been administered an anti-Claudin-1 antibody; and
(b) comparing the level of soluble CD147 in the test sample to a level of soluble CD147 in a control sample;
wherein a level of soluble CD147 in the test sample that is not increased relative to the level of soluble CD147 in the control sample indicates that the subject is susceptible to an increased dose of the anti-Claudin-1 antibody.

Embodiment 12. A method for treating a subject suffering from fibrosis, the method comprising the steps of:
determining whether the subject has increased levels of soluble CD147 by obtaining a test sample from the subject and comparing the level of soluble CD147 in the test sample to a control sample,
wherein the subject has previously been administered a dose of an anti-Claudin-1 antibody,
wherein if the subject has increased levels of soluble CD147 in the test sample relative to the control sample, then the subject is not administered an increased dose of the anti-Claudin-1 antibody;
wherein if the subject does not have increased levels of soluble CD147 in the test sample relative to the control sample, then the subject is administered an increased dose of the an anti-Claudin-1 antibody.

Embodiment 13. A method for designing a customized therapy for a subject suffering from fibrosis which comprises
(a) quantifying the level of soluble CD147 in a test sample from the subject, wherein the subject has previously been administered a dose of an anti-Claudin-1 antibody;
(b) comparing the level of soluble CD147 in the test sample obtained in step (a) with the level of soluble CD147 in a control sample, wherein if the level of soluble CD147 in the test sample obtained in step (a) is not increased relative to the level of soluble CD147 in the control sample, then the subject is susceptible to receive an increased dose of the anti-Claudin-1 antibody;
(c) determining that the subject has a not increased level of soluble CD147 with respect to the level of soluble CD147 in the control sample; and
(d) administering an increased dose of the anti-Claudin-1 antibody.

Embodiment 14. A method of classifying a subject suffering from fibrosis into a cohort and treating said subject, comprising
(a) quantifying the level of soluble CD147 in a test sample from the subject, wherein the subject has previously been administered a dose of an anti-Claudin-1 antibody;
(b) comparing the level of soluble CD147 in the test sample obtained in step (a) with the level of soluble CD147 in a control sample, wherein if the level of soluble CD147 in the test sample obtained in step (a) is not increased relative to the level of soluble CD147 in the control sample, then the subject is susceptible to receive an increased dose of the anti-Claudin-1 antibody;
(c) determining that the subject has a not increased level of soluble CD147 with respect to the level of soluble CD147 in the control sample;
(d) classifying said subject into a cohort based on the level of soluble CD147 in the test sample relative to the control sample; and
(e) administering an increased dose of the anti-Claudin-1 antibody.

Embodiment 15. The method of any one of embodiments 3, 4, 10, or 11, wherein if the subject is susceptible to an increased dose of the anti-Claudin-1 antibody, then the anti-Claudin-1 antibody is administered to the subject.

Embodiment 16. The method of any one of embodiments 2, 3, 9, or 10, wherein the anti-Claudin-1 treatment is an anti-Claudin-1 antibody

Embodiment 17. The method of any one of embodiments 1-16, wherein the anti-Claudin-1 antibody is administered intratumorally, intravenously, intraperitoneally, intramuscularly, intrathecally or subcutaneously.

Embodiment 18. The method of any one of embodiments 1-17, wherein the fibrosis is kidney fibrosis, lung fibrosis, or liver fibrosis.

Embodiment 19. The method of any one of embodiments 1-18, wherein the control sample is a sample from the subject prior to being administered an anti-Claudin-1 antibody or an anti-Claudin-1 treatment.

Embodiment 20. The method of any one of embodiments 1-8, or 15-19, wherein the level of soluble CD44 in the control sample is about 200 ng/mL to about 300 ng/mL.

Embodiment 21. The method of any one of embodiments 9-19, wherein the level of soluble CD147 in the control sample is about 600 ng/mL to about 800 ng/mL.

Embodiment 22. The method of any one of embodiments 1-21, wherein the dose of the anti-Claudin-1 antibody prior to being increased is 0.3 mg/kg or 3 mg/kg.

Embodiment 23. The method of any one of embodiments 1-21, wherein the dose of the anti-Claudin-1 antibody after being increased is 3 mg/kg, 10 mg/kg, or 20 mg/kg.

Embodiment 24. The method of any one of embodiments 1-23, wherein the anti-Claudin-1 antibody is a monoclonal antibody comprising the six complementarity determining regions (CDRs) of an anti-Claudin-1 monoclonal antibody secreted by a hybridoma cell line deposited at the DSMZ on Jul. 29, 2008 under an Accession Number DSM ACC2938.

Embodiment 25. The method of any one of embodiments 1-24, wherein the anti-Claudin-1 antibody is humanized.

Embodiment 26. The method of any one of embodiments 1-25, wherein the anti-Claudin-1 antibody comprises a VH comprising the amino acid sequence set forth in SEQ ID NO: 3 or SEQ ID NO: 13.

Embodiment 27. The method of any one of embodiments 1-26, wherein the anti-Claudin-1 antibody comprises a VL comprising the amino acid sequence set forth in SEQ ID NO: 4 or SEQ ID NO: 14.

Embodiment 28. The method of any one of embodiments 1-27, wherein the anti-Claudin-1 antibody comprises a VH comprising the amino acid sequence set forth in SEQ ID NO: 3; and a VL comprising the amino acid sequence set forth in SEQ ID NO: 4.

Embodiment 29. The method of any one of embodiments 1-28, wherein the anti-Claudin-1 antibody comprises a VH comprising the amino acid sequence set forth in SEQ ID NO: 13; and a VL comprising the amino acid sequence set forth in SEQ ID NO: 14.

Embodiment 30. The method of any one of embodiments 1-29, wherein the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

Embodiment 31. The method of any one of embodiments 1-30, wherein the anti-Claudin-1 antibody comprises a complementarity determining region (CDR) L1 comprising the amino acid sequence set forth in SEQ ID NO: 8, a CDR L2 comprising the amino acid sequence GA, and a CDR L3 comprising the amino acid sequence set forth in SEQ ID NO: 10.

Embodiment 32. The method of embodiment 1, wherein if the level of soluble CD44 in the test sample is not increased relative to level of soluble CD44 in the control sample, then the subject is administered nintedanib or pirfenidone.

Embodiment 33. The method of embodiment 8, wherein if the level of soluble CD147 in the test sample is not increased relative to level of soluble CD 147 in the control sample, then the subject is administered nintedanib or pirfenidone.

What is claimed is:

1. A method for identifying a subject having fibrosis that is suitable for therapy with an anti-Claudin-1 antibody or antigen binding fragment thereof, comprising the steps of:
a) obtaining a biological sample a human subject;
b) detecting the expression levels of one or more biomarkers selected from the group consisting of CD44, CD147, SPP1 and a combination thereof;
c) comparing the detected expression level of the one or more biomarkers with a control level of the one or more biomarker; and
d) identifying the human subject as a responder when the detected expression level of the one or more biomarkers is greater than the control level of CD44, CD147, or SPP1 expression,
thereby identifying the human subject having fibrosis that is suitable for therapy with the anti-Claudin-1 antibody or antigen binding fragment thereof.

2. The method of claim 1, comprising:
e) administering the anti-Claudin-1 antibody or antigen binding fragment thereof in an amount sufficient to alleviate a symptom of fibrosis when the subject is identified as a responder.

3. A method of identifying fibrosis progression in a human subject that is suitable for therapy with an anti-Claudin-1 antibody or an antigen binding fragment thereof comprising the steps of:
a) obtaining a biological sample a human subject;
b) detecting the expression levels of one or more biomarkers selected from the group consisting of CD44, CD 147, SPP1 and a combination thereof;
c) comparing the detected expression level of the one or more biomarkers with a control level of the one or more biomarker; and
d) identifying the human subject as a responder when the detected expression level of the one or more biomarkers is greater than the control level of CD44, CD147, or SPP1 expression,
thereby identifying fibrosis progression in the human subject that is suitable for therapy with an anti-Claudin-1 antibody or an antigen binding fragment thereof.

4. The method of claim 3, comprising:
e) administering the anti-Claudin-1 antibody or antigen binding fragment thereof in an amount sufficient to alleviate a symptom of fibrosis progression when the subject is identified as a responder.

5. A method for treating a subject having fibrosis, the method comprising the steps of:
a) obtaining a biological sample a human subject;
b) detecting the expression levels of one or more biomarkers selected from the group consisting of CD44, CD147, SPP1 and a combination thereof;
c) comparing the detected expression level of the one or more biomarkers with a control level of the one or more biomarker; and
d) identifying the human subject as a responder when the detected expression level of the one or more biomarkers is greater than the control level of CD44, CD147, or SPP1 expression, and
e) administering the anti-Claudin-1 antibody or antigen binding fragment thereof in an amount sufficient to alleviate a symptom of fibrosis when the subject is identified as a responder,
thereby treating the subject having fibrosis.

6. The method any one of claims 1-5, wherein the biological sample is derived from blood, scrum or tissue.

7. The method of any one of claims 1-6, wherein the control level of CD44 is about 200 ng/mL to about 400 ng/mL.

8. The method of any one of claims 1-7, wherein the control level of CD147 is about 400 ng/mL to about 800 ng/mL.

9. The method of any one of claims 1-8, wherein the control level of SPP1 is about 20 ng/mL to about 80 ng/mL.

10. The method of any one of claims 1-9, wherein the subject has previously been administered with anti-Claudin-1 antibody or antigen binding fragment thereof.

11. The method of claim 10, wherein the previous administration of anti-Claudin-1 antibody or antigen binding fragment thereof in the subject is administered with a first dose, and when the subject is identified as a responder, the subject is administered with at least a second dose of the anti-Claudin-1 antibody or antigen binding fragment thereof.

12. The method of claim 11, wherein the first dose and the second dose are equal.

13. The method of claim 11, wherein the second dose is greater than the first dose.

14. The method of any one of claims 11-13, wherein the first dose is about 0.3 mg/kg, 3 mg/kg, 10 mg/kg, 15 mg/kg or 20 mg/kg.

15. The method of claim 11-14, wherein the first dose administered one time, two times, three times, four times or five times.

16. The method of any one of claims 11-15, wherein the second dose is about 0.3 mg/kg, 3 mg/kg, 10 mg/kg, 15 mg/kg or 20 mg/kg.

17. The method of claim 11-16, wherein the second dose is administered one time, two times, three times, four times or five times.

18. The method of any one of claims 1-17, wherein the anti-Claudin-1 antibody or antigen binding fragment thereof comprises:
a CDR H1 comprising the amino acid sequence of SEQ ID NO: 5,
a CDR H2 comprising the amino acid sequence of SEQ ID NO: 6,
a CDR H3 comprising the amino acid sequence of SEQ ID NO: 7,
a CDR L1 comprising the amino acid sequence of SEQ ID NO: 8,
a CDR L2 comprising the amino acid sequence of GA, and
a CDR L3 comprising the amino acid sequence of SEQ ID NO: 10.

19. The method of any one of claims 1-18, wherein the anti-Claudin-1 antibody or antigen binding fragment thereof comprises a heavy chain variable region comprising the amino acid sequence of SEQ ID NO: 3 and a light chain variable region comprising the amino acid sequence of SEQ ID NO: 4.

20. The method of any one of claims 1-19, wherein the anti-Claudin-1 antibody or antigen binding fragment thereof comprises a heavy chain comprising the amino acid sequence of SEQ ID NO: 1 and a light chain comprising the amino acid sequence of SEQ ID NO: 2.

21. The method of any one of claims 1-20, wherein the anti-Claudin-1 antibody or antigen binding fragment thereof is a humanized antibody.

22. The method of any one of claims 1-21, wherein the anti-Claudin-1 antibody or antigen binding fragment thereof is administered intratumorally, intravenously, intraperitoneally, intramuscularly, intrathecally or subcutaneously.

23. The method of any one of claims 1-22, wherein the fibrosis is kidney fibrosis, lung fibrosis, or liver fibrosis.

24. The method of any one of claims 1-23, wherein the method comprises administering a therapeutically effective amount of at least one additional therapeutic agent, wherein the additional therapeutic agent is nintedanib or pirfenidone.

25. The method of claim 24, wherein the anti-Claudin-1 antibody or antigen binding fragment thereof and the at least one additional therapeutic agent is administered concurrently or sequentially.


SEQUENCE LISTING (27 sequences)

SEQ ID NO: 1 - protein, 448 aa, synthetic construct
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 119 aa, synthetic construct
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSS

SEQ ID NO: 4 - protein, 107 aa, synthetic construct
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIK

SEQ ID NO: 5 - protein, 8 aa, synthetic construct
GFSFSSYG

SEQ ID NO: 6 - protein, 8 aa, synthetic construct
ISPSGSYF

SEQ ID NO: 7 - protein, 12 aa, synthetic construct
ARLPGFNPPFDH

SEQ ID NO: 8 - protein, 6 aa, synthetic construct
QNVGGN

SEQ ID NO: 9 - skipped

SEQ ID NO: 10 - protein, 9 aa, synthetic construct
LQYKNNPWT

SEQ ID NO: 11 - protein, 448 aa, synthetic construct
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 214 aa, synthetic construct
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, synthetic construct
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSS

SEQ ID NO: 14 - protein, 107 aa, synthetic construct
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIK

SEQ ID NO: 15 - RNA, 5431 nt, Homo sapiens
  source 1..5431: mol_type = mRNA
ctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacaga

SEQ ID NO: 16 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 17 - RNA, 1594 nt, Homo sapiens
  source 1..1594: mol_type = mRNA
agcggttggaggttgtaggaccggcgaggaataggaatcatggcggctgcgctgttcgtgctgctgggattcgcgctgctgggcacccacggagcctccggggctgccggcacagtcttcactaccgtagaagaccttggctccaagatactcctcacctgctccttgaatgacagcgccacagaggtcacagggcaccgctggctgaaggggggcgtggtgctgaaggaggacgcgctgcccggccagaaaacggagttcaaggtggactccgacgaccagtggggagagtactcctgcgtcttcctccccgagcccatgggcacggccaacatccagctccacgggcctcccagagtgaaggctgtgaagtcgtcagaacacatcaacgagggggagacggccatgctggtctgcaagtcagagtccgtgccacctgtcactgactgggcctggtacaagatcactgactctgaggacaaggccctcatgaacggctccgagagcaggttcttcgtgagttcctcgcagggccggtcagagctacacattgagaacctgaacatggaggccgaccccggccagtaccggtgcaacggcaccagctccaagggctccgaccaggccatcatcacgctccgcgtgcgcagccacctggccgccctctggcccttcctgggcatcgtggctgaggtgctggtgctggtcaccatcatcttcatctacgagaagcgccggaagcccgaggacgtcctggatgatgacgacgccggctctgcacccctgaagagcagcgggcagcaccagaatgacaaaggcaagaacgtccgccagaggaactcttcctgaggtggcccgaggacgctccctgctccacgtctgcgccgccgccggagtccactcccagtgcttgcaagattccaagttctcacctcttaaagaaaacccaccccgtagattcccatcatacacttccttcttttttaaaaaagttgggttttctccattcaggattctgttccttaggtttttttccttctgaagtgtttcacgagagcccgggagctgctgccctgcggccccgtctgtggctttcagcctctgggtctgagtcatggccgggtgggcggcacagccttctccactggccggagtcagtgccaggtccttgccctttgtggaaagtcacaggtcacacgaggggccccgtgtcctgcctgtctgaagccaatgctgtctggttgcgccatttttgtgcttttatgtttaattttatgagggccacgggtctgtgttcgactcagcctcagggacgactctgacctcttggccacagaggactcacttgcccacaccgagggcgaccccgtcacagcctcaagtcactcccaagccccctccttgtctgtgcatccgggggcagctctggagggggtttgctggggaactggcgccatcgccgggactccagaaccgcagaagcctccccagctcacccctggaggacggccggctctctatagcaccagggctcacgtgggaacccccctcccacccaccgccacaataaagatcgcccccacctccaccctca

SEQ ID NO: 18 - protein, 269 aa, Homo sapiens
MAAALFVLLGFALLGTHGASGAAGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 19 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 15 aa, synthetic construct
GGGGSGGGGSGGGGS

SEQ ID NO: 21 - protein, 18 aa, synthetic construct
GSTSGSGKPGSGEGSTKG

SEQ ID NO: 22 - protein, 45 aa, synthetic construct
TTTPAPRPPTPAPTIASQPLSLRPEACRPAAGGAVHTRGLDFACD

SEQ ID NO: 23 - protein, 24 aa, synthetic construct
IYIWAPLAGTCGVLLLSLVITLYC

SEQ ID NO: 24 - protein, 42 aa, synthetic construct
KRGRKKLLYIFKQPFMRPVQTTQEEDGCSCRFPEEEEGGCEL

SEQ ID NO: 25 - protein, 447 aa, synthetic construct
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 26 - RNA, 1519 nt, Homo sapiens
  source 1..1519: mol_type = mRNA
agcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgccta

SEQ ID NO: 27 - protein, 300 aa, Homo sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN